(19) United States
(12) Patent Application Publication
(10) Pub. No.: US 2019/0134228 A1
(43) Pub. Date: May 9, 2019

(54) System and Method for Managing All Cancers by Disabling the Cancer Cells Ability to Reproduce

(71) Applicant: Richard Postrel, Miami Beach, FL (US)

(72) Inventor: Richard Postrel, Miami Beach, FL (US)

(21) Appl. No.: 15/808,563

(22) Filed: Nov. 9, 2017

(51) Int. Cl. A61K 49/00; A61K 9/50; A61K 35/763; A61K 35/761; A61K 35/765; A61K 35/76; C12N 15/113; A61K 38/02

(52) U.S. Cl. CPC A61K 49/0002; A61K 9/5015; A61K 35/763; A61K 38/02; A61K 35/765; A61K 35/76; C12N 15/113; A61K 35/761

(57) ABSTRACT

This invention provides tools and methods that prevent a cancer cell from growing and reproducing more cancer cells. When growth ceases the body's immune defenses are enabled to attack and destroy these cells if the cancer cell itself has not initiated its own natural apoptotic self-destruction processes. The tools and methods of the invention obstruct the metabolic adaptions required to support cancer growth. By addressing the increased rates of metabolism characteristic of all rapidly reproducing cancer cells using chemical and/or physical nanotechnology to identify, segregate, isolate these hypermetabolizing cells, the body's immune system and other natural defenses are empowered to further isolate and eliminate the diseased cells. The extreme growth rates required for their rapid reproduction involve massively increased rates of the biochemical reactions supporting the cancerous growth. Each excess reaction produces extra heat and raises the internal cell's temperature and the tissue space immediate to the rapidly growing cells. This heat signature is used as a primary biomarker that enables binding of a nanoviral particle engineered to migrate to at attach at the target site at the site and prevent the cell from continued metabolism. Preferably, the nanoparticle not only binds and blocks external membrane receptors on the target cell, but incorporates into the rapidly metabolizing cells additional metabolic blocking agents to stop their growth. When cell growth and proliferation are stopped, the body's natural defenses are able to segregate and eliminate these cells. The massively increased rates of metabolic reactions characteristic of cancer cells also produce excess acid. The decreased pH is useful as a secondary or confirmatory marker for identifying these cancer cells.

Cancer cells distinguish themselves from normal cells by their high rate of growing and reproducing new cells. The extreme growth rates required for their rapid reproduction involve massively increased rates of the biochemical reactions supporting the cancerous growth. Each excess reaction produces extra heat and raises the internal cell's temperature and the tissue space immediate to the rapidly growing cells. This heat signature is used as a primary biomarker that enables binding of a nanoviral particle engineered to migrate to at attach at the target site at the site and prevent the cell from continued metabolism. Preferably, the nanoparticle not only binds and blocks external membrane receptors on the target cell, but incorporates into the rapidly metabolizing cells additional metabolic blocking agents to stop their growth. When cell growth and proliferation are stopped, the body's natural defenses are able to segregate and eliminate these cells. The massively increased rates of metabolic reactions characteristic of cancer cells also produce excess acid. The decreased pH is useful as a secondary or confirmatory marker for identifying these cancer cells.

Cells are living things. As do all living things cells must conform to laws of chemistry and physics and we have applied laws of nature to describe how living things live by and apply these laws. One accepted law is that a living thing can reproduce or self-replicate, while lifeless entities regardless of their past do not self-replicate. Humans, like other sexually reproduced organisms begin as one single cell. As we grow and develop this originating cell must grow and produce daughter cells which differentiate, grow and proliferate to produce additional daughter cells continuously throughout our lifetimes. When the growth and proliferation processes become uncontrolled we call this disease phenomenon cancer.

Cancer cells, like all cells operate as a type of factory reliant on thousands of chemical reactions. These reactions tend to be exothermic and nominally warm the cell and its surrounding tissues. Since cancer cells grow faster than their parent cells, they have a higher heat signature. The accelerated growth rate makes the locally increased temperature common to all cancer cells. The metabolic requirements for rapid proliferation of daughter cells cause normal metabolic paths in cancer cells to shift ATP production in ways that increase H⁺and reduce ambient pH when the H⁺ and lactate counterion are exported to interstitial space.

The present invention exploits the characteristic local heat signature that occurs in conjunction with decreased local pH to identify, segregate, isolate and trigger natural elimination of these abnormally hyperproliferating cells.

### BACKGROUND

The American Cancer Society cites a 2014 study reporting direct medical costs for treating cancer in the US at $87.8 billion. An NIH study estimates by 2020 these costs will reach $158 billion when the total economic impact is estimated to be in the range of $1.5 trillion. The monetary and emotional costs to each cancer patient, their associates and their families are very severe. At its root each cancer involves a natural human cell that has unsuitably adapted its metabolic paths to support its rapid growth and proliferation of daughter cancer cells which beget increasing generations of maladapted cancer cells.

Growth and proliferation is essential for individual human development and legacy of our species. Like everything, this growth and development procession is not always perfect. One common deleterious anomaly involves uncontrolled cell divisions or hyperproliferation of a lineage of cells in our bodies. We characterize these anomalies as cancers.

Cancer is not a single disease, but cancers are a class of diseases each of which presents a metabolism that has been shifted to support the hyperproliferation that is characteristic of the cancer group. Although different cancers may appear in disparate tissues and cancer cells may migrate from one tissue to another, at their root each cancer cell cohort involves a shift in normal metabolism so that rather than supporting duties to maintain survival of the host organism the cell had differentiated to perform, the cancer cell's metabolism alters pathways, down-regulating several, up-regulating others, to improve the cancer cell's [undesired] hyperproliferative activities.

As an example, each cell division requires another set of nucleic acids to construct a second complete genome. The nucleic acid production pathway must be up-regulated. But the up-regulation of this one pathway will deprive other pathways of their normal resource pools. In view of these considerations, cancer can be thought of as a single disease—counterproductive hyperproliferation with several modes of expression dependent of the initial metabolic status of the cell and the adaptive switches or pressures modifying the initial metabolism to support hyperproliferation. Human life requires our cells to proliferate, but proliferation of our cells must be kept in balance. The present invent addresses the problem of hyperproliferation in two ways: 1) cells adapting their metabolisms along a path towards uncontrolled proliferation are provided stimuli to redirect them to more normal metabolisms, and 2) cells whose metabolisms cannot be reverted towards normal are stimulated to halt an essential metabolic pathway, to self-destruct or to be targeted for attack by the immune system.

To accomplish this we must consider that humans have learned to change or apply laws of nature in places or in ways that nature has not. But bottom line, while humans may be able to create copies or clones of things, including living things, only living things can self replicate without assistance.

An animal body, for example a human body, while not self-replicating an identical copy is able to reproduce additional members of the species. Most larger organisms have grown to develop a particularly useful tool of sexual reproduction. Sexual reproduction allows a species to experiment with various mixtures of life traits to increase the probability that at least several will survive a stress that if directed at a single ubiquitous essential trait might be capable of eliminating the entire species. The complex human organism has tens of thousands of genes serving different functions at different times. These genes can be turned on and turned off in accordance with chemical and physical laws. As an organism we have developed biochemical tools, enzymes, transcription factors, epigenetic markers, etc. that may operate at appropriate and preferably not inappropriate times to optimize survivability of that set of biochemical tools. By having slightly different mixtures within several adaptations of multiple traits (biochemical expression), members of a species who survive the stress may survive to reproduce more species members with the surviving tool set being available for participation in sexual reproduction. So over time, especially at times of severe stress, many versions of the tools in the set may not be adequate for many organisms to pass their biochemical tools on to the next generation. Only the most appropriate or most fit for the purpose of surviving the stress will survive. And not all variants are optimal at all times especially for different cell types or changed internal or external environments. So during the evolutionary process surviving organisms have developed coping mechanisms that have alterable pathways to accomplish the same or similar tasks using different available substrates, for example, and/or to respond to different intercellular signals.

Under a survival of the fittest rule, species must adapt to remain fit to successfully compete in varying environments. Since only organisms still living can successfully reproduce, species survival benefits from diverse capabilities of individuals. Being able to combine characteristics in different mixes from surviving members of the species has resulted in sexual reproduction evolving as a strong enhancer of species survival. But evolution also must involve change. Simply recombining what is there already cannot continue to produce the necessary changes and diversity. If no variants were being made, then over time all the original variations would have been selected out under circumstances when the current stress situation was not kind to that variant. Diversity would be destroyed and the species would have no ability to adapt to environmental shifts. Hence: mutation. But whole organisms cannot mutate, only cells that will grow and associate to build the organism can change their genetic material (mutate).

Mutation within the cell is necessary for species survival. But why fix a perfectly running machine? Biology has no mind. It just follows laws of physics and chemistry. While most times what has survived will be expected to continue to be survivable, in the long term, some aberration has to be accepted. We have seen that often mutation events increase when stress is present. This makes sense that in times where experimentation is desired to handle a changing (stressing) situation tools to cope with and overcome the stress would be more in use. So every time a cell makes a copy of itself, evolution dictates that depending on the level of difficulty the living thing is undergoing, changes in the genetic material (mutations) will occur in response. When a DNA strand is duplicated millions of chemical reactions are involved. It would seem unreasonable for each of these to be executed to perfection. Biology in fact has provided tools that can read and connect mismatches, gaps, duplications or insertions in the cell's genome. These tools are available for simple chemical mistakes as well as for foreign biologic entities attempting to replace or co-opt the host cell genome. Therefore most copy mistakes are corrected or eliminated. However, a fraction of mistakes and several segments of foreign genetic material are maintained in a dividing cell.

One area where mutation markers are well documented is in the study of Inborn Errors of metabolism (IEMs). Over a century ago Archibald Garrod popularized the concept that human diseases were inheritable in accordance with rules of Mendelian genetics. More than 500 IEMs have now been catalogued including some apparently symptom free, but perhaps showing alternative metabolites to specific substrate sources; others may lead to early death. Several IEM disease, such as Glut1 deficiency and phenylketonuria result from mutations that prevent the relevant gene's expression in an active form. These two diseases, if detected before severe physiologic damage, can be managed by limiting the availability of the molecules handled by these proteins.

In fact, recessively inherited loss-of-function mutations in enzymes and transporters constitute the bulk of IEMs. IEMs and most other results of mutation events are classified as “diseases” because they decrease the probability of the carrier of the mutation successfully reproducing. These mutations in germ line cells would face elimination unless the defect is addressed in an alternative manner (e.g., a different pathway, a different environment).

While most times our cells, including germ line cells, but also somatic cells, faithfully copy the DNA genetic material to replicate new cells, as part of evolution our genetic material has been selected to be very, very, slightly unfaithful. In individuals aging is correlated with mutated genomic material. Most mutations do not lead to cancer, but in rare but still significant cases mutations start a cell down a hyperproliferative pathway that may eventually present as a cancer. In 2015 the median age of a human with a cancer diagnosis was 66 years.

This is relevant to cancer. In cancer a group of cells presents a group of mutations. But cancer itself is not naturally in our genetic material. A specific group of cancer genes is not suddenly switched on. Cancer cells are living things and therefore follow chemical and physical laws and the principles of biology. Cancer itself is a complex disease. A cancer cell is not different in just a single respect from normal desirable cells. Many events are necessary to develop all the changes that make a cell cancerous.

Not every mutation improves survivability. Many mutations result in a non-functioning gene that if other features cannot compensate adequately for will mean that that cell will not survive. Biological systems have evolved to preferentially take out poorly functioning cells. One important process in this regard is apoptosis. Apoptosis is a process that has evolved to remove undesirable cells. For example, apoptosis is triggered to remove cells at the base of baby teeth to facilitate disposal when adult teeth are coming in. Apoptosis may selectively remove cells at times of stress. For example, several cells may be sacrificed during lean times to preserve nutrition for remaining cells. Cells that misfunction for one reason or another, for example become leaky to Ca⁺⁺, will present metabolic abnormalities. Many of these abnormalities increase probability of cell death through mitosis.

But some mutations survive. Within the body, each cell, though guided by evolution, tries to survive. As a result, over a lifetime, several mutations in each cell can be expected to occur and to be carried through to daughter cells. As the cells continue to operate, many of the cells will harbor mutations. Some mutations may be silent; some mutations may be quiescent (not turned on, but available if stimulated). But all will be passed on when this cell divides. So what makes a cancer cell?

Cancer cells have been altered to follow a metabolic program to enhance necessary biosynthesis and support that cell's proliferation. The changes may not be in the best interests of the organism. But concomitant with these metabolic changes must be changes that evade the organism's control of inappropriately behaving cells and that evade the apoptotic cell death protocols carried in each cell's genetic instruction set.

One notable change in rapidly proliferating cells in general, but in cancer cells in particular there is a metabolic switch from using the mitochondria for efficient production of adenosine triphosphate (ATP) to favor the less energetically efficient cytoplasmic pathway for ATP production. This alterative pathway produces less ATP per glucose molecule and finishes with lactate, a three carbon molecule, as a chemically energetic metabolite that must be excreted. This requires a protein to transport the lactate ion across the cell membrane. Lactate is transported by one of several monocarboxylate transporter proteins (MCTs).

As mitochondrial ATP production is de-emphasized cytoplasmic pathways using enzymes evolved for those pathways become more active. Generally, expression is accentuated, often at the transcription level, and carrying through messenger RNA to ribosomal synthesis of extra protein copies.

Some proteins are up regulated. Others are down regulated. Many will feedback or regulate activity of other functions or cell proteins. For example, pyruvate kinase M2 (PKM2) plays a part in the altered glucose metabolism characteristic of cancer. When pyruvate kinase M2 (PKM2) interacts with phosphotyrosine-containing proteins it inhibits the enzyme's activity, resulting in an increased availability of glycolytic metabolites the cell may use to support cell proliferation. An alternate, pyruvate kinase M1 (PKM1), same gene but processed differently within the cell, does not share this outcome. It can therefore be said that favoring conditions that increase PKM2 at the expense of PKM1 is one factor supporting cancer development. While a mutation in the pyruvate kinase gene itself may affect splicing, a mutation in another gene or even an extracellular signal turning on another path within the cell may be part of this cell's path to cancer.

As cells collect mutations, many will be culled. But occasionally a cell presenting a mutation leaning towards cancer cell metabolism will continue to reproduce. Several of the reproductions may be additionally mutated. The same stress that may have encouraged the premiere mutation may encourage subsequent mutations and/or the premiere (or a subsequent) mutation may provide added stress encouraging still more mutations. Many of these mutations will be removed by the organism's survival processes, but in rare, but significant to the organism, occasions multiple mutations may increase survivability of that cell line and continue to proliferate with continuously expanding mutations carried in the cell line's genome. At some point the collection of mutations will be sufficient to escape organismal control and will favor proliferation over the function the organism would like that cell type to perform.

### SUMMARY OF THE INVENTION

Cancer cells present as a disease characterized by a detrimental expression of numerous traits, particularly traits leading to a rapid cell division. A cell's life can be defined as the sum of all chemical reactions occurring in the cell. Since cancer cells differ from normal cells, their chemical reactions (aka metabolism) must by definition also differ.

The present invention exploits the metabolic adaptations a cancer cell requires for its specialized cancerous metabolism to identify, segregate, isolate and trigger natural death in these abnormally hyperproliferating cells.

Cancer cells arise from diverse tissues and from many, many cell types, but at the root of any cancer is that cell's increased rate of making new cells, that is: hyperproliferation. Every time a cell proliferates it splits to create two cells each of which requires its own membrane, cytoskeleton, nucleus, mitochondria and other organelles. This duplication requires the cell to accelerate synthetic pathways and several additional pathways that support accelerated synthesis. The resulting two cells will require a doubling of DNA for duplicated nuclei, additional membrane lipids and proteins to cover the increased surface/volume ratio, extra endoplasmic reticulum, golgi, mitochondria, lysosomes, etc. to be split between two cells during mitosis. Mitosis itself is a resource hungry process requiring a slew of catabolic and anabolic events. In essence a metabolic push is necessary to provide an additional set of all cellular components and the temporary resources and energy necessary to divide the cell into two. This accentuated metabolism can be employed to guide intercourse between an interested party and the cancerous or precancerous metabolically modulated cell(s).

Regardless of the cell type originating the cancer, all cancer cells will present an increased uptake of nutrient building blocks into the cell, increased use of the nutrients (reactants) in various chemical reactions to make increased products. The products will include products useful for sustaining the cell and by-products such as waste chemicals and heat. While there are some common chemical waste products of metabolism, one ubiquitous product (since in general metabolism is exothermic) is an increased heat output.

Since cancer cells produce more heat than surrounding cells, increased temperature is a marker that can be used to identify and target these cells. While monitoring local temperature is not essential for all means of attacking cancer metabolism, heat can serve as a trigger or signal activating or making available an anti-cancer therapy. The cells essentially self-identify though their cancer adapted metabolisms. Many physical or chemical tools that measure or monitor temperature are available to identify the cells or zones of cells with cancer associated hypermetabolic states. On a micro-or nano scale, electronic and/or chemical sensors can be made to accumulate at locations or at cell membranes that are responsible for characteristics such as increased temperature and decreased pH. Using specific characteristics of the hyperproliferating cancer cells allows these cells to be segregated from normally metabolizing cells and tissues. By localizing with the targeted cells the cell or zone of cells chemical or physical sensor compounds or components can isolate the targeted cells from healthy tissue cells and instigate one or more of several natural paths of these cells to their growth arrest and cell death. The isolated cells may be restrained by many possible interventions including, but not limited to: nutrient deprivation, membrane disruption, viral infection, mitochondrial autophagy, mitotic arrest, apoptosis stimulation, transcription alteration or cessation, interference RNA, etc.

The identification, segregating, isolating and triggering can include mechanics that are very high tech. For example, nanoparticles can be configured with nano-sensor capabilities. These nano-particles can be supplied in the vicinity of a tumor or may be applied more systemically, such as in blood or lymph vessels. One species of particle we can make has a form of nano-motor, or means of moving itself. These can be random or can be configured to be thermotaxic (move towards or away from a heat source) or chemotaxic (move along a chemical gradient, such as a pH gradient). Phototaxic (responsive to light—electromagnetic radiation, radio waves) sensors are another example, but these would be effective only close to the skin using ambient light or as secondary sensors responsive to a primary sensor that directs the secondary sensor to an identified location. Nanoparticles can also be configured as receivers of electromagnetic radiation. Nanoparticles compartmentalized for example by physical and/or chemical means can be queried to confirm location and if desired about the particle's surroundings. For example, the particle may report back an indication of temperature, pH, and or other parameter programmed into the sensor. When the sensor is configured as an antenna, electromagnetic energy can be converted to heat energy at the target location.

As mentioned above, a sensor nanoparticle may also be a reporter nanoparticle, a courier nanoparticle and/or a signal nanoparticle able to deliver a preprogrammed substance or to recruit other couriers for delivery when a preprogrammed event is reported. Nanoparticles can be mostly physical in their action, may include chemical elements to aid in sensing or for delivery and may even transport biologic cargo(es) depending on the whims of the nanoparticles creator(s).

Less technological applications of the invention are also available. Chemicals, especially lipid compositions, are heat responsive. Many chemical reactions are temperature dependent. Thermo-dependence is even more evident in enzymatic reactions where subtle temperature changes can induce profound changes in a protein's (enzymes are most often proteins) or RNA's (some RNAs (ribonucleic acids) have enzymatic or binding/presenting activities) folding (3-dimensional structure) and activity.

We have several decades experience using temperature to change nucleic acid binding and activity and are adept at engineering sequences to fold or unfold at desired temperatures. Nucleic acids can be engineered to produce a protein of interest, including proteins whose range of temperatures where they are active is an engineering consideration, using available and improving software. Nucleic acids whose transcription, processing and/or translation are required to make the proteins can also be engineered for desired temperature dependence.

Another feature common to the metabolic shift of cancer cells is decreased reliance on the electron transport chain for making high energy phosphates, e.g., adenosine triphosphate (ATP). To make the ATP, which is required in amplified amounts to support the increased metabolism necessary for the hyperproliferation, cells step up a glycosylation process that leaves lactate and hydrogen ion (H⁺ ) as a byproduct. The additional H⁺ lowers the pH (a measurement of H⁺ concentration). Another common byproduct is various reactive oxygen species (ROS) such as H₂O₂ and O₂⁻. Target specificity may be improved through use of a plurality of altered metabolism indicators and/or switches for activating one or more metabolic modulators, including cell death modulators. For example, a probe or effector sensitive to both temperature and pH, e.g., a) with bind-sensitive movement and/or binding and H⁺ lability, b) an effector molecule that partitions according to pH gradient whose activity is temperature dependent, c) a first component partitioned according to pH or temperature and a second component with temperature or pH dependent binding to the first, etc. are examples of taking advantage of two or more manifestations of altered metabolic states to effect metabolic changes in the targeted zones, e.g., rebalancing cells' metabolisms and/or initiating cell death in cells incapable of restoration.

These chemical signatures can be use in addition to or as alternative to the heat signature given off by cancer cells for identification and targeting. pH can also be used as an activator or triggering mechanism extracellularly and/or intracellularly. Lactate may serve as a surrogate or confirmatory indicator of pH. An agent engineered to bind the cancer cell may bind, for example, lactate and/or lactic acid after export or when still associated with the producing cell. For example, he practitioner may elect to use a nanoparticle engineered to bind lactate and its transporter at the cell membrane.

ROS species are very reactive and therefore would have great applicability when used as an intracellular activator, but in specific circumstances these can be used as an activator signal or as a switch signal to be amplified in an extracellular application. For example, a sensor particle may be activated by an ROS at a relevant pH.

Although not observed in every cancer cell type, the increased metabolism results in a modified plasma membrane. Some modifications are for stability, such as slightly longer fat chains in the membrane to compensate for the increased heat of metabolism. Most cells also have increased membrane transporters; some cancer cells express binding or transport proteins not normally expressed in the neighboring more properly differentiated cells. In other instances, a transporter is found at elevated concentrations in the membrane to support the substantially increased needs to transport some raw nutrients. While these may be available as secondary targeting or trigger mechanisms, the primary mechanism, increased need for certain chemical reactions within the hyperproliferating cell, is the fundamental mechanism underpinning the identifying, targeting mechanisms of this invention.

Any available targeting or delivery means known in the art can be used. For example, a viral particle can be engineered to deliver a therapy to the targeted cell's interior. In the example of a reovirus which infects cells that express an activated ras oncogene, the cell is rendered more prone to infection by the virus since the activated Ras system deactivates antiviral defenses the cell would normally use to prevent reovirus infection. An engineered retrovirus, like a reovirus, or other vector known in the art is therefore a viable courier for a variety of therapeutic strategies to modulate intracellular metabolism especially when anti-viral defenses are compromised as often occurs when a cell ramps up its proliferative capacity.

Viral re-engineering has been a niche but is now a growing art. For example, Asokan et al, Nature biotechnology, volume 28: 1, Jan. 2, 2010, 79-82, teaches reengineering the receptor ligand of adeno-associated virus, with special emphasis on a basic [non-acidic] hexapeptide stretch at positions 585-590. (Charge and/or polarity of a peptide segment correlates positively with its availability for binding.) The engineered adeno-associated virus is defective in replication, requiring coinfection with another virus such as adenovirus, HSV, etc.

Madigan and Asokan, Current Opinion in Virology, Volume 18, June 2016, Pages 89-96 summarizes progress in engineering adeno-associated viral binding character. The glycan surface having been mapped, with multiple serotypes identified, isolated and characterized, bases for selecting optimal adeno-associated vectors is well-developed. “A thorough structural understanding of AAV capsid glycan interactions has enabled rational manipulation of glycan footprints on the AAV capsid surface. This re-engineering approach has yielded novel, synthetic AAV strains with potential applications in therapeutic gene transfer. Specifically, structure-inspired design has been utilized to abrogate capsid binding to glycan receptors, alter binding affinity, and more recently engineer orthogonal glycan receptor interactions.” Multiple exemplary re-engineering successes are briefly mentioned in the paper along with a summary statement: “A thorough structural understanding of AAV capsid glycan interactions has enabled rational manipulation of glycan footprints on the AAV capsid surface. This re-engineering approach has yielded novel, synthetic AAV strains with potential applications in therapeutic gene transfer. Specifically, structure-inspired design has been utilized to abrogate capsid binding to glycan receptors, alter binding affinity, and more recently engineer orthogonal glycan receptor interactions.” In this and other peer reviewed papers the adeno-associated virus is set forth as an advantageous candidate for vector re-engineering.

Non-reproducing constructs such as synthetic vesicles, e.g., liposomes, are another therapeutic option. The vesicular membranes can be engineered to be sensitive to heat, pH, ROS or other chemical attractant or binding agent.

Nanoparticles, including nanosensorparticles can also be employed as couriers. The particles may be coated to facilitate targeted binding. They may include another binding moiety to secure a second binding agent that may act as a diagnostic flag or may carry therapeutic substance.

In the case of virus, strains of vaccinia virus, herpes virus, vesicular stomatitis virus, senaca virus, Semliki Forest virus, ECHO or REGVIR virus, and monstrously attenuated polio virus have been similarly tested and characterized in cancer cells or in animals or in humans with cancers for their inherent cell killing effects, primarily targeted at cancers.

For best efficiency the couriers will preferably transport a molecule whose effects are multiplied at or in the cell. For example, the courier may carry: RNAi with downstream effects on one or more of the cell's pathways, transcription factors, methylation factors, demethylation factors, an engineering cassette such as used in CRISPR/cas, a plasmid that can infect mitochondria, a ligand that opens a pore in an organelle such as the nuclear membrane or mitochondrial membrane, packets that increase expression of a protein or group of proteins to favor or disfavor one or more metabolic pathways (such as the electron transport pathway of mitochondria), mitochondrial fusion or fission modulators, anti-apoptotic or pro-apoptotic compounds such as Bcl or Bad, etc.

Antisense RNA was recognized over 30 years ago as a means for suppressing synthesis from a complementary mRNA. However, the early attempts in using these to suppress expression showed unacceptable off-target effects. Improvements including double stranded RNAs have been recognized to have near universal effect in most cells of multicellular organisms and as such can provide a focal mechanistic system for the regulation of mRNA function. Many derivations are known in the art and are not repeated here.

Viruses naturally function by vectoring genetic material into cells they co-opt to produce more viral particles. Several viral genuses have had members engineered and used for treating cells. One genus is lentiviruses. Lentiviruses are a genus of viruses of the Orthoretrovirinae subfamily within the Retroviridae family. Members of this genus include pathogens of bovine, equine, feline, ovine, and primate receptor targets. Lentiviruses are enveloped viral particles that bud from an infected cell's plasma membrane. Viral particles are 80 to 120 nm in diameter, containing a single-stranded 9.2-kb RNA genome and several structural proteins, including the matrix, capsid, nucleocapsid, envelope, and reverse transcriptase enzymes. Lentivectors feature efficient transduction of especially nondividing cells, minimal natural anti-vector immunity in human hosts, and a low potential for genotoxicity resulting from insertional mutagenesis. Several modifications of the lentivector have improved their safety profile and ability to elicit a strong immune response. Viral particles bind to their target cell through the targeted cell's receptor and the virus's envelope glycoprotein. The particle fuses with the plasma membrane releasing the genomic RNA into the cell's cytoplasm where it is reverse transcribed to double stranded DNA on its path to incorporation in a host chromosome.

In the 1980s retroviral particles were used to deliver therapeutic genes. Since these experiments, issues of viral particle instability, inability to transduce non-dividing cells and low titers have been addressed, e.g., by using engineered lentiviruses. Further engineering efforts including, but not limited to: elimination of viral genes for Vpr, Vif, Nef, and Vpu; replacing the Tat and 5′LTR with a constitutive promoter and moving Rev onto a different plasmid have improved safety and efficiency. Additional engineered features include, but are not limited to: adding woodchuck hepatitis B posttranscriptional regulatory element (WPRE) to improve gene expression; deleting the U3 region of the 30 LTR to generate self-inactivating transfer vectors (SINS); and including a triple-helix signal (TRIP) to improve nuclear import.

Specificity for host cells is engineered by modifying envelope proteins or transgene expression promoters. Vesicular stomatitis protein is one example for broadening the host repertoire. This or other stand-in gene can be engineered for pH and/or temperature selectivity. Such engineered lentiparticles have been used to vaccinate an organism and to induce cell suicide in targeted cells. Since the lentiparticle fuses with the plasma membrane such particles are suitable vectors for introducing various molecules including, but not limited to: siRNA, microRNA, snoRNA, lincRNA, a ribozyme, piRNA, double stranded and long double stranded ncRNA.

Vaccinia viruses, especially the Lister strain, have been engineered rather successfully for selective binding and host cell infection. DNA, of select size up to about 25 kb can be included in the genome. By selecting a host cell and host cell conditions for producing viral particles the envelope lipid content can be engineered for selective fusing. These vectors are not beholden to any specific surface receptor, but directly fuse with the targeted cell membrane. Singly enveloped particles directly fuse with targeted membranes to release particle contents into the host cell cytoplasm. In a doubly enveloped format the particle is engulfed in an endocytotic process and the low pH cleaves the outer envelope allowing the inner envelope to fuse with the endosome membrane and release contents to the cytoplasm. Vaccinia can be engineered for selective, e.g., heat sensitive lipid envelope, pH sensitive envelope, selective lipid content etc. By selecting the threshold energy for fusion through propagating cell selection and/or engineering, vaccinia can be engineered for wider or narrower selectivity.

The absence of required protein-protein interaction found in other viral infection processes allows for broad selection of cells to be infected, while membrane lipid and glycolipid control is a valuable and wide range engineering opportunity. Another advantage of the lipid-lipid interfacing for fusion is that if a targeted cell membrane protein is altered, replaced or absent in the aberrant cell that is being targeted, the lipid interfacial binding is negligibly affected.

Vaccinia from other species are generally attenuated or extremely attenuated in their ability to reproduce in humans. While immune suppressed or immune compromised individuals should be considered as higher risk patients, spot testing and monitoring may be all the additional care that is necessary for these individuals. An extremely attenuated version of a pox virus has been created for congenital immune deficient and for immune failing (e.g., AIDS) patients. By avoiding human and/or primate derived cells for proliferating the viral particles, the extreme attenuation can be maintained while other features such as select glycolipid and lipid membrane content are engineered in.

Herpes simplex virus 1 (HSV-1) has properties that render it ideal for engineering into a selectively replicating vector for targeting tumors or other undesired or foreign cells. These advantages include, but are not limited to: a large non integrating genome that includes multiple nonessential genes, a potent cytolytic potential, an adapted capacity to evade the immune system, etc. HSV-1 is a large, naturally neurotropic, double-stranded DNA virus that can become and remain latent using a stable episomal element in the targeted cell. Timing of activity is controllable using the antiviral compound, gangcyclovir. Convection-enhanced delivery (CED) implants fine catheters, OD less than about 1 mm, directly into the targeted region or zone. Cells infected with HSV become targets for immune system elimination , which elimination can be accelerated using an anti-viral drug like acyclovir.

Reoviruses have special advantage when targeting transformed cells. Reovirus type 3 Dearing is a double-stranded RNA virus that is ubiquitous and nonpathogenic in humans. It has been shown to be oncolytic in its propensity to replicate in transformed cells but not in normal cells. Reovirus is only active when the Ras pathway has suppressed other activities, especially dsRNA-activated protein kinase (PKR). In cells with activated Ras, PKR is not phosphorylated and thus remains unable to mitigated reoviral attack.

Wild type reovirus gains cell entry through endocytosis after attaching to the cell through junctional adhesion protein (JAM-1) as a prelude to endocytosis. For specificity towards pH depressed cells, the reovirus can be altered through generations of growth and selectivity (similar to attenuation) to take on a modified receptor specificity for example for the lactate export protein(s) MHC1 and/or MHC4. A degree of temperature selectivity is obtainable through careful control of lipid content of the proliferative host cell when reovirus is produced.

Reoviral infection marks infected cells for immune attack so no suicide gene insert into the reoviral genome is absolutely mandated, but engineered reogenome may be modified to augment immune system targeting or may be engineered to compromise cell growth and proliferation through other means. There is some evidence that simple reoviral infection may render the cells sensitive to increased temperature. Though this temperature related death may be synergistically augmented by the body's immune system. A reovirus selected for preferential binding to an MHC or other acid related protein, possibly with an envelope facilitating endocytosis preferentially infects cells whose activated ras is part of the cancerous transformation. Allowing the reovirus to deliver a bio-substance inhibitory of toxic to the cell can accelerate the cell's elimination with or without requiring immune system intervention. Allowing the reovirus to self-replicate can amplify this effect in neighboring cells.

Adenoviral related vectors have a long history in gene therapeutic endeavors. Wild type adenovirus has selectivity for upper respiratory tract cells expressing coxsackievirus-adenovirus receptor (CAR). However, susceptibility can be expanded as adenovirus has been successfully modified to attack cells infected with other viruses that leave a membrane imprint. EB virus, herpes simplex virus, other adenovirus, Newcastle disease virus, reovirus, vaccinia virus, Sindbis virus, etc., maybe used in conjunction with modified adenovectors to doubly tag targeted cells.

Adeno associated virus cell access involves a two stage process. The first stage comprises attachment to a primary receptor. This is followed by interaction with a secondary receptor to accomplish the internalization. Adenovirus enters cells in a clathrin-coated vesicle for transport to endosomes, where acidification results in partial disassembly of the capsid. The partial virion proceeds into the cytoplasm and is transported to the nucleus for its replication.

This two-stage entry mechanism renders the viruses open to a large variety of primary receptors. The major function of the primary (fiber) receptor is to hold the virion adjacent to the cell surface to encourage and enable its interaction with an integrin molecule. So a variety of cell surface molecules can serve this function and the virion can be modified using standard genetic engineering tools to manage its affinity for a desired plasma membrane protein, for example, a residual protein from a previously admitted virus.

All cells are living entities and as living things they require raw materials to maintain function, to grow and to reproduce. Multiple tissues, helper cells, cell organelles and nutrients all affect each cell's viability differentially depending on the cells association with these factors and the cell's adapted metabolism.

Lone cells can obtain their nutrition from the immediate surroundings. But in complex organisms, where the cell may be distant from the outside environment a delivery service is necessary. In larger animals the circulatory system is responsible for delivering and clearing food and waste. A blood supply transgressing through a system of tubes (blood vessels) is used. As the organism grows each part must be supplied with appropriate blood vessels for support. The formation of blood vessels requires migration and proliferation of endothelial cells. These endothelial cells must be fueled in order to form and maintain the circulatory system.

The circulatory system is also an information system. Blood can carry chemical messages to and from the cells it services. The message does not need a locational address. Since cells are in contact with the environment (interstitial space) they are constantly removing chemicals from the space and depositing chemicals into it. The tools on the cell surface that help transport chemicals across the cytoplasmic membrane are exposed to the interstitial space. If a molecule has characteristic affinity for one of these “receptors” it will associate as a ligand with the receptor. A receptor may have one of many functional characteristics. It may serve to allow viral attachment to the cell membrane. It may act enzymatically to change the ligand in a manner including, but not limited to: isomerization, cleavage, covalent attachment, internalization (carry across the membrane), initiate encapsulation, present the ligand in receptive form to another ligand or receptor, etc. The receptor often will induce further changes inside the cell to manage (or metabolize) in some way the molecule being brought into the cell. While often signals are molecules manufactured by one cell and delivered to another to instruct that cell what it should do, simply classical food molecules can serve as signals to upregulate the pathways needed to metabolize that type of molecule.

Most cells ingest the chemical mass and energy they need to grow and proliferate in a form of carbon they find easy to use, e.g., amino acids (proteins) and sugars (carbohydrates).

However, when the cell is behaving in a specialized manner, the cell often must alter its pathways to support the specialized needs. Or in the chicken-egg question, when the cell has activated surprising metabolic pathways, then the cell will by necessity be doing something distinct from “normal” cells.

For example, a growth signaling receptor protein when activated will cascade a signal through to the cell nucleus to build food receptors and to transport these receptors to the plasma membrane. A sugar or amino acid maybe contacts the receptor and is carried inside. The transporter will initiate an appropriate pathway inside the cell to metabolize the cargo. Perhaps the cargo is aminated or otherwise modified to divert to a less common metabolic pathway or to serve as an intracellular signal.

One popular branching point, i.e., a molecule that might be directed through several pathways is acetyl Co-A. Often acetyl co-A is produced from the degradation of carbohydrates and/or proteins. But, especially in circumstances where nucleic acid synthesis is required (e.g., rapidly proliferating cells or cells expanding mitochondrial mass) fatty acids may become a favored source of carbon.

Acetyl-CoA is a lipogenic precursor for many lipid molecules including, but not limited to: isoprenoid, cholesterol and fatty acids.

Oxaloacetic acid, which may also be directly exported from the TCA cycle from the mitochondria, supplies pools of non-essential amino acids.

Mitochondria are organelles in cells that are best known for production of ATP from electron transfer (oxidation/reduction) reactions. The size and shape of mitochondria can vary within a single cell and each mitochondrial package may contain plural copies of the mitochondrial genome, a double stranded circular DNA that encodes 37 genes. Mitochondria are dynamic organelles that can migrate within a cell along cytoskeleton framework. Mitochondria can grow by fusing with other mitochondria and may dissociate in a process termed fission that allow split up smaller bodies to move more freely. The smaller mitochondria produced through fission have reduced distance for diffusion. Mitochondria can grow by adding additional membrane and protein materials and may be digested through a process termed mitophagy or autophagy. In general, smaller bodies will have better communication with the cytoplasm due to reduced volume to surface ratio.

One target of cancer treatment could theoretically involve hindering the ability of cancer cell mitochondria to participate in either of these fusion or fission processes and thereby impact general mitochondrial functioning. However, accelerating the fission process in comparison to fusion may be one means through which neoplastic cells can diminish their death through apoptosis. Maintaining joined mitochondria as favored by fusion processes appears to make an apoptotic event more possible. Several proposed rounds for use in practicing the present invention emphasize maintenance of fused mitochondria. Mitochondria in cells are consistently changing. They are transported along the cytoskeleton to areas of need. They may change from more rodlike to more spherical shapes depending on location within a cell. During these processes, mitochondria may fuse together and may split apart under control of proteins within the cell. Two mitochondrial membrane proteins essential for mitochondrial fusion are mitofusin 1 (Mfnl) and mitofusin 2 (Mfn2) which connect two mitochondrial membranes as the fusion process begins. On the other side, another essential protein for maintaining healthy mitochondria is Drpl, a primarily cytosolic protein. When bound to a mitochondrion, Drp1 forms a constrictive ring around a mitochondrion to split it into two parts. Drp1 is one of the GTPase proteins in mammalian cells. Drp1 interacts with several proteins including, but not limited to: Fis1, Mff, MiD49 and MiD51, that act on the mitochondrial surface to initiate and control mitochondrial fission. Fission is important for maintaining a healthy mitochondrial population and appears to be necessary for cells to proliferate. Drp1 activated mitochondrial fission is associated with inhibiting apoptosis, a property opposite that of eliminating the individual cell. Thus interfering with activity of any of these proteins may slow fission and maintain mitochondria in a fused state. Cancer cells are characterized by relatively fewer fused mitochondria with respect to more independent or smaller separate mitochondria than seen in non-malignant cells. Consistent with this observation is a finding that Drp1 expression is elevated in cancer cells and that the fraction of Drp1 phosphorylated at the serine residue at position 616 in Drp1, activated Drp1, is elevated. Apparently, cancer cells increase phosphorylation at this spot with the effect of favoring fission activities. It is possible to chemically inhibit fission by interfering with Drp1. Mitochondrial division inhibitor 1 (Mdivi 1) is a quinazolinone derivative that selectively inhibits mitochondrial division by blocking dynamin GTPase activity in mammalian cells (IC50=˜50 μM). It has been shown to prevent apoptosis by inhibiting mitochondrial outer membrane permeabilization in vivo and to block Bid-activated Bax/Bak-dependent cytochrome c release from mitochondria in vitro. Cayman Chemical reports that Mdivi 1 has been used to maintain mitochondrial integrity and to prevent cell death in models of pathological conditions including cancer, heart failure, and ischemia and reperfusion injuries. Another inhibitor of Drp1 is a compound known as P110. The polypeptide P110, DLLPRGT, appears more selective for blocking Drp1/Fis1 interaction than Drp1 interaction with other ligands. [A novel Drp1 inhibitor diminishes aberrant mitochondrial fission and neurotoxicity. Xin Qi, Nir Qvit, Yu-Chin Su, Daria Mochly-Rosen. J Cell Sci 2013 126: 789-802; doi: 10.1242/jcs.114439.] Delivering one or more Drp1 inhibitors in a cocktail to the cancer cell targets can potentiate other pro-apoptotic interventions.

Pyruvate Kinase M2 Activators Promote Tetramer Formation and, Suppress Tumorigenesis

Cancer cells are distinguished from other cells usually based on their loss of controlled functions normally carried out by that organ or cell type and by hyperproliferation. While the hyperproliferation can be understood from the viewpoint of the cell whose life mission is to grow and continue its cell lineage, from the organism's point of view this group of rogue cells is not supportive of the life of the large organism. First, these cells are not performing activities for the good of the whole organism, second, these cells are wasting nutrients, third, the increased volume occupied by these cells interferes with communication and other functions of the non-cancer cells, fourth, these cells are consuming (wasting) resources that could be more advantageously used, and fifth, these cells may be exporting toxic or problematic metabolites requiring surrounding tissues to expend resources and effort in clean-up operation.

Since the cells are performing different, i.e., abnormal, activities one would expect that reactions within cancer cells will be differ from normal cells. To put it simply, different outputs and behaviors will require different activities to achieve them. The hyperproliferative action of the mutating or mutated will require abundance of nutrients. The increased rate of reactions will produce excess metabolites, possibly abnormal metabolites and will result in excess heat from the exothermic reactions which predominate in the general nature of reactions.

The cells will also differ in the way they utilize intracellular and extracellular nutrients. Addressing these differences provides strategies for impeding tumor growth and tumor cell proliferation. For example, as the cells hyperproliferate pathways for manufacturing purines and pyrimidines for nucleic acids must be accelerated.

Enhanced glucose uptake is a hallmark of several cancers and has been exploited in the clinic as a diagnostic tool through PET imaging of the glucose analogue 18F-deoxyglucose (18FDGPET). Moreover, in contrast to most normal tissues where much of the glucose is oxidized through the TCA cycle, in mitochondria, cancer cells preferentially convert glucose to lactate a three carbon molecule that retains and eventually removes energy unavailable for ATP synthesis. The fate of glucose inside cells is influenced by the enzymatic properties of the specific glycolytic gene products expressed. Expression of the M2 isoform of pyruvate kinase (PKM2) can contribute to the characteristic glucose metabolism of tumors and replacement of PKM2 with its splice variant PKM1 cannot efficiently support biosynthesis and tumor growth. Pyruvate kinase appears to be an important gateway in glucose metabolism that can be critical for controlling cell proliferation.

The aversion to the electron transport chain and the conventional oxidative phosphorylation pathway should not be considered an anomaly of cancer cells. Remembering that these cells were once considered “normal” cells but in their progression to the hyperproliferative state have had to alter normal cell functions, the hyperproliferation would be expected to change some metabolic pathways to support the new activities. These abnormal pathways would be expected to require abnormal raw materials in the nutrients consumed or in the metabolic intermediates necessary to sustain the new way of life for the cell. It is thus wise to think of the altered metabolism, not as a symptom of cancer, but as links in the causative chain.

Most cancers are believed to present with a genetic abnormality. Several genes have alleles that support or initiate development of cancer. An external event switching a gene on or off may initiate the cancer cascade. Many viruses have now been shown to increase cancer risk following their insertion into the victim cells' genome. If the organism is inattentive to the changing cell, the cell may be allowed to continue development to a cancerous status. But to support the change the cell will have to adapt. Some adaptations will be built in, in accordance with feedback loops that evolution has given us; some may involve additional mutations in the nuclear or mitochondrial genomes; some may be more complex evolved responses, for example, an epigenetic modification like methylation.

As the base is metabolism and the nutrition supporting the metabolism, the variety of underlying causes and adaptations may require a variety courses to counter the metabolic signature of a cancer cell.

One course of treatment will be to support “normal” metabolism. That is to provide raw material (nutrients) supporting normal metabolism, for example to favor electron transport chain activity. In concert with this can be a restriction on types of raw materials supporting the diverted or cancer enhanced or enhancing metabolic pathways. A more aggressive strategy may include inhibitors of one or more of these side pathways. When these cells are deprived of the environment in which they mutated and may have in fact contributed to, selective pressure will tilt against these cells in favor of the “normal” cells.

Nutrition can also be altered with a goal of supporting apoptotic activity and inhibiting cells that counter apoptosis.

Besides simply altering nutrition, in many cases the gene expression will have been irreversibly altered. These modifications, whether in primary sequence or epigenetic modifications offer a grand opportunity for treatment. Genetic engineering tools can recognize specific mutations and when coordinated with an endonuclease can remove or edit identified genetic abnormalities. Systems such as CRISPR have recognized ability to distinguish methylated from non-methylated bases in genetic sequence.

Gene editing processes are continually being improved. To date they have improved precision and specificity and become acceptable in practice. An example of a recent summary of CRISPR technology appears in US patent Application 20170035860.
Gene editing technologies: Recent developments of technologies to permanently alter the human genome and to introduce site-specific genome modifications in disease relevant genes lay the foundation for therapeutic applications in CNS disorders such as Parkinson's disease (PD) or Alzheimer disease (AD). These technologies are now commonly known as “genome editing.” Current gene editing technologies comprise zinc-finger nucleases (ZFN), TAL effector nucleases (TALEN), and clustered regularly interspaced short palindromic repeats (CRISPR)/CRISPR-associated (Cas) system or a combination of nucleases (e.g. mutated Cas9 with Fok1) (Tsai, S. Q., Wyvekens, N., Khayter, C., Foden, J. A., Thapar, V., Reyon, D., Goodwin, M. J., Aryee, M. J., and Joung, J. K. (2014). Dimeric CRISPR RNA-guided Fold nucleases for highly specific genome editing. Nature biotechnology 32, 569-576.)) All three technologies create site-specific double-strand breaks. The imprecise repair of a double strand break by non-homologous end joining (NHEJ) has been used to attempt targeted gene alteration (nucleotide insertion, nucleotide deletion, and/or nucleotide substitution mutation). A double-strand break increases the frequency of homologous recombination (HR) at the targeted locus by 1,000 fold, an event that introduces homologous sequence at a target site, such as from a donor DNA fragment. Another approach to minimize off-target effects is to only introduce single strand breaks or nicks using Cas9 nickase (Chen et al., 2014; Fauser et al., 2014; Rong et al., 2014; Shen et al., 2014).

The CRISPR/Cas9 nuclease system can be targeted to specific genomic sites by complexing with a synthetic guide RNA (sgRNA) that hybridizes a 20-nucleotide DNA sequence (protospacer) immediately preceding an NGG motif (PAM, or protospacer-adjacent motif) recognized by Cas9. CRISPR-Cas9 nuclease generates double-strand breaks at defined genomic locations that are usually repaired by non-homologous end-joining (NHEJ). This process is error-prone and results in frameshift mutation that leads to knock-out alleles of genes and dysfunctional proteins (Gilbert et al., 2013; Heintze et al., 2013; Jinek et al., 2012). Studies on off-target effects of CRISPR show high specificity of editing by next-generation sequencing approaches (Smith et al., 2014; Veres et al., 2014) (FIG. 1, panel 1).

Other applications for heart disease, HIV, and Rett syndrome have been described. (Ding et al., 2014; Swiech et al., 2014; Tebas et al., 2014). For heart disease, permanent alteration of a gene called PCSK9 using CRIPR technology reduces blood cholesterol levels in mice (Ding et al., 2014). This approach was based on the observation that individuals with naturally occurring loss-of-function PCSK9 mutations experience reduced blood low-density lipoprotein cholesterol (LDL-C) levels and protection against cardiovascular disease (Ding et al., 2014). A second example for the feasibility of this approach is HIV. Individuals carrying the inherited Delta 32 mutation in the C—C chemokine receptor type 5, also known as CCR5 or CD195 are resistant to HIV-1 infection. Gene modification in CD4 T cells were tested in a safety trial of 12 patients and has shown a significant down-regulation of CCR5 in human (Tebas et al., 2014). Another recent study showed the successful use of CRISPR/Cas9 technology in CNS in a mouse model for the editing of the methyl-binding protein 2 (MecP2) gene. Mutation in this gene causes Rett syndrome, a condition in young children—mostly girls—with mental retardation and failure to thrive. In this approach an adeno-associated virus (AAV) was used as the delivery vehicle for the Cas9 enzyme in vivo. Overall, 75% transfection efficiency was described with a high targeting efficiency that almost completely abolished the expression of MecP2 protein and functionally altered that arborization of the neurons similar to what has been described for Rett syndrome (Swiech et al., 2014). This shows the proof of concept that gene editing using CRISPR/Cas9 technology is achievable in the adult brain in vivo.

Despite reports in the literature describing the use of genetic editing techniques, none have been described or suggested for genes associated with neurodegenerative disorders. A strong need continues to exist in the medical arts for a method for treating and/or inhibiting diseases associated with neurodegenerative disorders, such as materials and techniques useful for the treatment of Parkinson's Disease.

### SUMMARY OF THE INVENTION

In a general and overall sense, the present invention provides for the arrest and/or prevention of neurodegeneration associated with neurodegenerative disease in vivo. In some embodiments, arrest and/or prevention of neurodegeneration is accomplished using gene editing methodologies and molecular tools to manipulate specific gene(s) and/or gene regulatory elements, to provide a modification of the gene and/or genomic regions associated with neurodegeneration and neurodegenerative disease, such as Parkinson's Disease.

In some aspects, the present invention provides a method of treating a neurological deficit associated with neuropathological disease comprising administering a genetically engineered vector comprising a gene for a nuclease and a promoter for the nuclease, as well as an appropriate molecular “guide” into a cell. Following the administration, the vector facilitates an expression of a molecular component that alters a gene in the cell or expression of a targeted gene associated with the neuropathology in the cell. The affected gene would be implicated in an etiology of the neurological deficit.

In other embodiments, a medical composition for treating a neurological deficit in a patient is provided. The medical composition includes a nuclease that introduces double strand break in a gene implicated a neurological deficit, a guide RNA that targets a gene implicated in neurological disease, and a delivery system that delivers the nuclease and guide RNA to a cell.

For purposes of the description of the present invention, the term “modification of gene and/or genomic region” may be interpreted to include one or more of the following events (FIG. 1):

a) Targeted introduction of a double-strand break by a composition disclosed, resulting in targeted alterations (random mutations e.g. insertions, deletions and/or substitution mutations) in one or more exons of one or more genes. This modification in some embodiments provides a permanent mutation in a cell or population of cells having the modified gene.

b) Targeted binding of non-functional mutant Cas9 to non-coding regions (e.g. promoters, evolutionary conserved functional regions, enhancer or repressor elements). Binding is induced by compositions disclosed. Sterical hindrance of binding of other proteins (e.g. transcription factors, polymerases or other proteins involved in transcription) may also result as a consequence of binding.

1. CRISPR sgRNA introduces small insertions or deletions through non-homologous end joining (NHEJ), in general several nucleotides, rarely larger fragments (Swiech et al., 2014).

2. Homology-directed repair (HDR) to correct point mutations by introducing a non-natural, but partially homologous template.

3. Double Genome editing of splice-sites or splicing related non-coding elements to eliminate certain gene regions, e.g. exon 5 of SNCA gene.

4. Double Genome editing of non-coding or intronic gene regions to eliminate regulatory elements that increase or decrease gene expression, e.g. D6 or 112 regulatory region in SNCA gene.

5. sgRNA guides mutant Cas9 to physically inhibit binding of transcription factors in promoter region,

6. sgRNA guides mutant Cas9 to physically inhibit binding of transcription factors in regulatory regions or intronically.

Gene editing or modification can be achieved by use of any variety of techniques, including zinc-finger nuclease (ZFN) or TAL effector nuclease (TALEN) technologies or by use of clustered, regularly interspaced, short palindromic repeat (CRIPSR)/Cas9 technologies or through the use of a catalytically inactive programmable RNA-dependent DNA binding protein (dCas9) fused to VP16 tetramer activation domain, or a Krueppel-associated box (KRAB) repressor domain, or any variety of related nucleases employed for gene editing. These can be seen as existing tools to sever the genomic region in question.

The tools mentioned above, are general in their application. Aspects of the present methods and compositions provide the design of custom CRISPR single-guide RNA (sgRNA) sequences specific for coding gene regions and regulatory sequences in genes implicated in neurodegeneration. In this manner, an exact genomic location for precise gene alteration in humans may be accomplished, with a resulting improvement and/or elimination of a neurodegenerative disorder pathology or symptom.

Additional patents and patent applications, for example, US application no. 20170015994 evidence the utility, feasibility and enablement of gene editing processes with high specificities are well known and accepted in the art.

These genetic abnormalities can thus be considered targets that are recognizable by some very specific tools. Different strategies are available for treating these cells. The DNA might simply be cut and irreversibly capped to prevent further mitosis by that cell. Incorrect genes might be turned on, for example, to initiate cell division before the genome had been copied, to activate genes incompatible with continued viability of the cell, to correct the gene abnormality, to permanently turn off the gene. These and similar strategies would increase stress on the cells, especially cells expressing the targeted genetic modification, and even if not fatal to the cell will reduce its fitness and survivability.

When a cell characteristic can be targeted, e.g., a Ras expressing cell targeted by a virus, the weapon might be factors to turn on, activate, augment, or duplicate activity of desired proteins. These can be proteins supporting and restoring more normal metabolism, but might also be proteins supporting cell death, for example proteins supporting initiation or progression of apoptosis. On the flip side, anti-apoptotic protein activity or expression might be blocked.

Transcription factors or other manipulation of transcription may be used to increase expression of a protein or to throttle it down. The targeted gene need not be a gene mutated in the cancer process, so long as the weapon is acceptably targetable. These might be protein or nucleic acid based and could be directed against a modified gene, of course, but can also be targeted against more ubiquitously required genes to accomplish a proliferation event. Genes involved in the cell cycle, genes involved in cytoskeleton, genes required for membrane integrity, etc., essentially any well used or essentially expressed gene might be selected for the ultimate target. RNAi can be used to inhibit transcription and therefore protein activity. DNA or modified DNAs might be incorporated into genomic material.

Pyruvate kinase catalyzes the last step of glycolysis, transferring the phosphate from phosphoenolpyruvate (PEP) to adenosine diphosphate (ADP) to yield adenosine triphosphate (ATP) and pyruvate. In mammals, two genes encode a total of four pyruvate kinase isoforms. The Pkrl gene encodes the PKL and PKR isoforms, expressed in the liver and red blood cells respectively. Either the PKM1 or PKM2 isoform encoded by the Pkm gene is found in cells. PKM1 is found in many normal differentiated tissues whereas the PKM2 is expressed in most proliferating cells including all cancer cell lines and tumors tested. PKM1 and PKM2 are derived from alternative splicing of a Pkm gene transcript by mutual exclusion of a single conserved exon that encodes 56 amino acids. Despite the similar primary sequences, PKM1 and PKM2 have different catalytic and regulatory properties. PKM1 appears always active, exhibiting high constitutive enzymatic activity. In contrast, PKM2 is less active, but is allosterically activated by the upstream glycolytic metabolite fructose-1,6-bisphosphate (FBP).

Unlike other pyruvate kinase isoforms, PKM2 can interact with proteins harboring phosphorylated tyrosine residues thereby releasing FBP which, in a feedback mechanism, reduces the activity of the enzyme. Low PKM2 activity, in conjunction with increased glucose uptake, facilitates use of glucose carbons into anabolic pathways derived from glycolysis. Also, PKM2, but not PKM1, can be inhibited by direct oxidation of its cysteine 358 as an adaptive response to increased intracellular reactive oxygen species (ROS).

Additionally, PKM2 expression in cancer cells has been associated with enhanced phosphorylation of the H₁₁ on phosphoglycerate mutase 1 (PGAM1) by PEP. This pathway is an alternative route for pyruvate production but bypasses the generation of ATP via the pyruvate kinase step. This supports high rates of glycolysis. Replacement of PKM2 with the constitutively active isoform PKM1 results in reduced lactate production, enhanced oxygen consumption, and a decrease in PGAM1 phosphorylation. There also appears to be selection for PKM2 expression for growth in vivo. Alternatively, PKM2 expression may evidence selection against high pyruvate kinase activity and therefore against expression of PKM1. This rationale suggests that activation of PKM2 may impede cancer cell proliferation by interfering with regulatory mechanisms critical for proliferative metabolism.

It is expected that PKM2 activators will mimic the regulatory properties of constitutively active PKM1, thereby promoting high PKM2 activity regardless of the known mechanisms cells use to decrease pyruvate kinase activity. Similar to results observed when PKM2 is replaced with PKM18, under standard tissue culture conditions, PKM2 activators had no significant effects on cell proliferation when tested across several lines. In contrast, when proliferation is assessed under hypoxic conditions (˜1% O₂), PKM2 activator treatment results in decreased rate of cell proliferation (in comparison to DMSO-treated cells). And expression of PKM1 in the presence of endogenous PKM2 has no effect on cell proliferation in standard tissue culture conditions, but inhibits proliferation under hypoxia to a similar degree as treatment with PKM2 activators. Replacement of PKM2 with PKM1 also impairs cell proliferation under hypoxic conditions.

Cancer cells harbor genetic changes that allow them to increase nutrient uptake and alter metabolism to support anabolic processes, and interfering with this metabolic program is a viable strategy for cancer therapy. Altered glucose metabolism in cancer cells is mediated in part by expression of PKM2, which has specialized regulatory properties. Unlike its splice variant PKM1, which is found in many normal tissues, PKM2 is allosterically activated by FBP and can interact with tyrosine-phosphorylated proteins to release FBP and decrease enzyme activity.

Thus, growth factor signaling promotes decreased PKM2 activity and availability of glycolytic metabolites for anabolic pathways that branch from glycolysis. This suggests that activation of PKM2 might oppose the effects of growth signaling and interfere with anabolic glucose metabolism.

In this situation where pyruvate kinase activation has occurred, high pyruvate kinase activity would suppress tumor growth.

### Mitochondrial Genome

Mitochondria have a limited set of genes in their genome. Most proteins in the mitochondrial membranes and matrix are encoded in the nuclear genome before being translated on the cytoplasmic ribosomes. These nuclear encoded mitochondrial genes include, but are not limited to: mitochondrial enzymes, mitochondrial membrane pore and carrier proteins and chaperone or folding proteins.

The mitochondrial genome consists of one double stranded DNA polymer in a circular format, i.e., no apparent beginning or end. Mitochondrial genes can code for RNA or polypeptide polymers. The 37 mitochondrial genes are split between the two complementary DNA strands. A strand with higher guanine cytosine ration is called the H-strand and the complement is dubbed the L-strand. The H-strand is richer in genes with twenty-eight of the thirty-seven. L-strand genes include TRNA, TRNC, TRNE, TRNY, TRNN, TRNP, TRNS1, ND6 AND CR; while the H-strand genes encode TRNT, CYTB, NDS, TRNL2, TRN2, TRNH, ND4, ND4L, TRNR, ND3, TRNG, COX3, ATP6, ATPS, TRNK, COX2, TRND, COX1, TRNW, ND2, TRNM, TRNI, ND1, TRNL1, RRNL, TRNV, RRNS AND TRNF.

A typical cell will contain between 10² and 10⁴ DNA molecules (paired strands). However, sex cells vary with the egg carrying ˜2×10⁵ and sperm bringing 10¹ or fewer. Typically perhaps 15 mitochondria may harbor up to 500 genome molecules total. But numbers vary with cell type and with time in a given cell.

Most mitochondrial genes encode molecules that remain in the mitochondrion. Only humanin (an anti-apoptotic protein) is mtDNA encoded (by the larger ribosomal RNA encoding gene), but human is exported from the mitochondrion and exerts its effects after release into the cytoplasm. The mitochondrion has its own ribosomal RNAs (2) and tRNAs (22). Leucine and Serine each have two tRNAs.

Mitochondrial proteins encoded by mitochondrial DNA (mtDNA) are involved in the electron transport chain which has five complexes: NADH:ubiquitone reductase, succinate dehydrogenase, cytochrome bc₁, cytochrome c oxidase and ATP synthase. Each of these complexes resides in the inner mitochondrial membrane.

Seven Complex 1 protein subunits are encoded in mtDNA: ND1, ND2, ND3, ND4, ND4L, ND5 and ND6. Thirty-eight additional protein complex subunits are encoded in the cell nucleus. Two copies of NDUFAB1 are in a complex 1 assembly, but every other subunit: NDUFA2, NDUFA6, NDUFA7, NDUFA12, NDUFS1, NDUFS4, NDUFS6, NDUFV1, NDUFV2, NDUFV3 (in the N module); NDUFA5, NDUFS2, NDUFS3, NDUFS7, NDUFS8 (in the Q module); MT-ND1, NDUFA3, NDUFA6, NDUFA13, (in the ND module); MT-ND2, MTND3, NDUFA1, NDUFA10, NDUFC1, NDUFC2, NDUFS5 (in the ND2 module); MT-ND4, NDUFB1, NDUFB5, NDUFB10, NDUFB11 (in the ND4 module); MT-ND5, NDUFB3, NDUFB7, NDUFB8, NDUFB9 (in the ND5 module); NDUFA11 (possibly bridging the Q and ND1 modules); and NDUFAB1, NDUFA9, NDUFB4, NDUFB6 (uncertain module assignment) is only present as a single copy. Complex 1 assembly is facilitated or chaperoned by assembly factors including, but not limited to: Ndufaf3 (C3orf60), Ndufaf4 (C6orf66), Ndufaf1 (CIA30), C20orf7, Ecsit, Ind1 and Ndufaf2 (B17.2L).

Other proteins encoded by nuclear DNA but transported into the mitochondria include but are not limited to: Phosphoenolpyruvate carboxykinase, Hinge protein (Fragment), 14-3-3 protein epsilon, Tryptophanyl-tRNA synthetase, VDAC4 protein (Fragment), Voltage-dependent anion-selective channel protein 3, Voltage-dependent anion channel (Fragment), Voltage-dependent anion-selective channel protein 2 , Voltage-dependent anion-selective channel protein 1, Vesicle-associated membrane protein 1 (VAMP-1) (Synaptobrevin 1), Ubiquinol-cytochrome C reductase complex 11 kDa protein, Ubiquinol-cytochrome C reductase iron-sulfur subunit, Ubiquinol-cytochrome C reductase complex core protein 2, Ubiquinol-cytochrome C reductase complex core protein I, Ubiquinol-cytochrome C reductase complex 14 kDa protein, Ubiquinol-cytochrome C reductase complex 7.2 kDa protein, Uracil-DNA-glycosylase, Uracil-DNA glycosylase, mitochondrial precursor (UDG), Mitochondrial uncoupling protein 4 (UCP 4), Mitochondrial uncoupling protein 3 (UCP 3), Mitochondrial uncoupling protein 2 (UCP 2) (UCPH), Mitochondrial brown fat uncoupling protein 1 (UCP 1) (Thermogenin), Thioredoxin reductase 2, Thioredoxin, Mitochondrial translation elongation factor EF-Tu (Fragment), Elongation factor Tu, Thiosulfate sulfurtransferase (Rhodanese), Elongation factor Ts, Heat shock protein 75 kDa, DNA topoisomerase I, Mitochondrial precursor proteins import receptor (Translocase of outermembrane TOM70), Probable mitochondrial import receptor subunit TOM40 homolog, Mitochondrial import receptor subunit TOM20 homolog , Probable mitochondrial import receptor subunit TOM7 homolog, Mitochondrial import receptor Tom22, Trimethyllysine dioxygenase, Thymidine kinase 2, Thymidine kinase, Mitochondrial import inner membrane translocase subunit TIM9 A, Mitochondrial import inner membrane translocase subunit TIM8 B, Mitochondrial import inner membrane translocase subunit TIM8 A, Import inner membrane translocase subunit TIM44, Mitochondrial import inner membrane translocase subunit TIM23, Mitochondrial import inner membrane translocase subunit TIM22, Mitochondrial import inner membrane translocase subunit TIM17 B (JM3), Mitochondrial import inner membrane translocase subunit TIM17 A, Mitochondrial import inner membrane translocase subunit TIM13 B, Mitochondrial import inner membrane translocase subunit TIM13 A, Mitochondrial import inner membrane translocase subunit TIM10, Tumorous Imaginal discs homolog precursor, Transcription factor 1, Putative ATP-dependent mitochondrial RNA helicase, Surfeit locus protein 1, Sulfite oxidase, Succinyl-CoA ligase [GDP-forming] beta-chain, Succinyl-CoA ligase [GDP-forming] alpha-chain, Succinyl-CoA ligase [ADP-forming] beta-chain, Steroidogenic acute regulatory protein, Single-stranded DNA-binding protein, Succinate semialdehyde dehydrogenase, Superoxide dismutase [Mn], Smac protein, Sodium/hydrogen exchanger 6 (Na⁺/H⁺ exchanger 6) (NHE-6), ADP/ATP carrier protein, liver isoform T2 (ADP/ATP translocase 3), ADP/ATP carrier protein, fibroblast isoform (ADP/ATP translocase 2), ADP/ATP carrier protein, heart/skeletal muscle isoform T1 (ADP/ATP translocase 1), Phosphate carrier protein, mitochondrial precursor (PTP), Mitochondrial 2-oxodicarboxylate carrier, Mitochondrial carnitine/acylcarnitine carrier protein, Mitochondrial deoxynucleotide carrier, Solute carrier family 25, member 18, Peroxisomal membrane protein PMP34, Mitochondrial ornithine transporter 1, Brain mitochondrial carrier protein-1 (BMCP-1), Calcium-binding mitochondrial carrier protein Aralar2, Calcium-binding mitochondrial carrier protein Aralar1, Mitochondrial 2-oxoglutarate/malate carrier protein (OGCP), Mitochondrial dicarboxylate carrier, Tricarboxylate transport protein, Serine hydroxymethyltransferase, Serine hydroxymethyltransferase, cytosolic, Sideroflexin 3, Sideroflexin 2, Sideroflexin 1, Oligoribonuclease, Succinate dehydrogenase (ubiquinone) cytochrome B small subunit, Succinate dehydrogenase cytochrome b560 subunit, Succinate dehydrogenase (ubiquinone) iron-sulfur protein, Succinate dehydrogenase (ubiquinone) flavoprotein subunit, Nonspecific lipid-transfer protein, SCO2 protein homolog, mitochondrial precursor, SCO1 protein homolog, mitochondrial precursor, Seryl-tRNA synthetase, Reticulon 4 (Neurite outgrowth inhibitor), 40S ribosomal protein S3a, Mitochondrial 28S ribosomal protein S21 (MRP-S21) (MDS016), 28S ribosomal protein S17, mitochondrial precursor (MRP-S17)(HSPC011), 28S ribosomal protein S16, mitochondrial precursor (MRP-S16) (CGI-132), 28S ribosomal protein S15, mitochondrial precursor (MPR-S15) (DC37), 2-5A-dependent ribonuclease, NADH dehydrogenase subunit 3 homolog/ND3 homolog (Fragment), Delta 1-pyrroline-5-carboxylate synthetase (P5CS), Serine protease HTRA2, Lon protease homolog, Proline oxidase, Peroxiredoxin 5, Thioredoxin-dependent peroxide reductase, Protoporphyrinogen oxidase, Peptidyl-prolyl cis-trans isomerase, Inorganic pyrophosphatase 2, DNA-directed RNA polymerase, DNA polymerase γ subunit 2, DNA polymerase γ-subunit 1, ARTS protein, Mitochondrial processing peptidase γ-subunit, Mitochondrial processing peptidase α-subunit, Paraplegin (Spastic paraplegia protein 7), Probable glutamyl-tRNA(Gln) amidotransferase subunit B, Phosphatidylethanolamine N-methyltransferase, Pyruvate dehydrogenase protein X component, [Pyruvate dehydrogenase [Lipoamide]]-phosphatase 2, [Pyruvate dehydrogenase [Lipoamide]]-phosphatase 1, [Pyruvate dehydrogenase [lipoamide]] kinase isozyme 4, [Pyruvate dehydrogenase [lipoamide]] kinase isozyme 3, [Pyruvate dehydrogenase [lipoamide]] kinase isozyme 2, [Pyruvate dehydrogenase [lipoamide]] kinase isozyme 1, Pyruvate dehydrogenase E1 component β-subunit, Pyruvate dehydrogenase E1 component α-subunit, testis-specific form, Pyruvate dehydrogenase E1 component α-subunit, somatic form, Pyruvate dehydrogenase E1-alpha-subunit (Fragment), Pyruvate dehydrogenase E1-alpha-subunit (Fragment), Pyruvate dehydrogenase E1-alpha-subunit (Fragment), Pyruvate dehydrogenase E1-alpha-subunit (Fragment), Pyruvate dehydrogenase E1-alpha-subunit (Fragment), Pyruvate dehydrogenase E1-alpha-subunit (Fragment), Peptide deformylase, Mitochondrial 28S ribosomal protein S30 (MRP-S30), Programmed cell death protein 8, Phosphoenolpyruvate carboxykinase, mitochondrial precursor [GTP], Phosphoenolpyruvate carboxykinase, cytosolic [GTP] , Propionyl-CoA carboxylase beta chain, Propionyl-CoA carboxylase α-chain, Pyruvate carboxylase, Transmembrane protein, Succinyl-CoA:3-ketoacid-coenzyme A transferase, Cytochrome oxidase biogenesis protein OXA1, Ornithine carbamoyltransferase, Mitochondrial ornithine transporter 2, Optic atrophy 3 protein, Dynamin-like 120 kDa protein, Mitochondrial outer membrane protein 25, N-glycosylase/DNA lyase, 2-oxoglutarate dehydrogenase E1 component, Ornithine aminotransferase, Nuclear respiratory factor-1 (NRF-1), NAD(P) transhydrogenase, Nucleoside diphosphate kinase, Neurolysin, mitochondrial precursor, NOGO-interacting mitochondrial protein, Cysteine desulfurase, NADH-ubiquinone oxidoreductase 9 kDa subunit, NADH-ubiquinone oxidoreductase 24 kDa subunit, 24-kDa subunit of complex I (Fragment), NADH-ubiquinone oxidoreductase 51 kDa subunit, NADH-ubiquinone oxidoreductase 23 kDa subunit, NADH-ubiquinone oxidoreductase 20 kDa subunit, NADH-ubiquinone oxidoreductase 13 kDa-A subunit, NADH-ubiquinone oxidoreductase 15 kDa subunit, NADH-ubiquinone oxidoreductase 18 kDa subunit, NADH-ubiquinone oxidoreductase 30 kDa subunit, NADH-ubiquinone oxidoreductase 49 kDa subunit, NADH-ubiquinone oxidoreductase 75 kDa subunit, NADH-ubiquinone oxidoreductase subunit B14.5b, NADH-ubiquinone oxidoreductase KFYI subunit, NADH-ubiquinone oxidoreductase B22 subunit, NADH-ubiquinone oxidoreductase ASHI subunit, NADH-ubiquinone oxidoreductase B18 subunit, NADH-ubiquinone oxidoreductase B17 subunit, NADH-ubiquinone oxidoreductase SGDH subunit, NADH-ubiquinone oxidoreductase B15 subunit, NADH-ubiquinone oxidoreductase B12 subunit, NADH-ubiquinone oxidoreductase AGGG subunit, NADH-ubiquinone oxidoreductase PDSW subunit , NADH-ubiquinone oxidoreductase MNLL subunit, Acyl carrier protein, NADH-ubiquinone oxidoreductase 39 kDa subunit, NADH-ubiquinone oxidoreductase 19 kDa subunit, NADH-ubiquinone oxidoreductase subunit B14.5a, NADH-ubiquinone oxidoreductase B14 subunit, NADH-ubiquinone oxidoreductase 13 kDa-B subunit, NADH-ubiquinone oxidoreductase MLRQ subunit, NADH-ubiquinone oxidoreductase B9 subunit, NADH-ubiquinone oxidoreductase B8 subunit, NADH-ubiquinone oxidoreductase 42 kDa subunit, NADH-ubiquinone oxidoreductase MWFE subunit, and NADH dehydrogenase subunit 6. These human proteins have had their genes sequenced and are known in the art.

Modifying a cell's nuclear DNA is actually more straight forward than modifying a cell's mitochondrial DNA simply because of the number of relevant genes in a cell. Nuclear genes have but two alleles, one allele on each half of the pared chromosomes. Mitochondrial genes in a single cell are much more abundant; an individual mitochondrion may have several dozen circular genomes; and each cell can have a dozen or more mitochondria. Because mitochondria are continuously fusing with other mitochondria each mitochondrion may include heteroplasmic copies. Many cancer cells present with homoplasmic mtDNA mutations. This suggests that the mutated mtDNA rendered significant survival benefits to the homoplasmicly mutated cell. Given the environment in the e.g., hyperproliferating cancer cell, the homoplasmic mitochondrial mutations when paired with the cell's other metabolic deviations were strong supporters of survivability of those mitochondria and of the cell hosting them.

Since the nucleus and mitochondria contain DNA as their genetic material similar genetic engineering principles can apply. Gene editing involves excising, inserting or substituting one or more genes or epigenetic modification of a gene, i.e., modifying a gene sequence or modifying ability of a transcription factor to bind and initiate or halt a gene's transcription.

Excising a gene will require the DNA molecule to be cleaved at the beginning and end of the DNA strand being removed. Insertion requires but one cleavage point with each end of the opening being compatible (usually short complementary overlapping single stranded endpoints). Substitution events require both excision and insertion. Sometime the excision and insertion sites are identical, but this is not an absolute requirement.

DNA molecules are nucleotide acids and are cleaved by nuclease enzymes (nucleases). Four classes of nuclease have been employed extensively in genetic engineering: meganucleases, zinc finger nucleases (ZFNs), transcription activator-like effector-based nucleases (TALEN), and the CRISPR-Cas system.

Gene editing systems can be made specific to mutated sequences, including epigenetic mutations. In cells then only undesired mutations could be made to serve as a check to prevent side effects on healthy cells. The recognition site might be used simply to allow correcting a single mutation, but given that the cancer process involves many events in many of the cell's compartments in many instances the mutation recognition will serve as confirmation for the vector to effect a fatal cleavage or to insert a DNA sequence designed to be fatal to the cell. In nuclear DNA success rates are expected to be higher because of the limited number of targets in each cell as compared to the multiple copies in each mitochondrion and the multiple mitochondria per cell. To further improve efficacy and to take into account the continuing change as cancers develop and mature multiple targets and/or multiple fatal outcomes can be programmed into the editing processes.

For mitochondria the sheer number is complicating. With so many copies of the genome and possibly multiple variants even in one mitochondrion, the task is more complex. First, the sheer number of copies suggests that rather than few or even a single editing machine being delivered to the cell, a self-replicating machine may be more effective. Once in the cell the editing tool would co-opt the cells machinery as viruses have learned to do to proliferate inside the cell in sufficient numbers to have desired effect. Unlike the difficulties presented in trying to correct a mitochondrial disease where the intent is to make multiple corrects to preserve the cell and other cells throughout the organism, generally this desired effect will be fatal to the cell.

One exploitable feature is that if a significant number of mitochondria are comprised the mitophagy/autophagy process, Ca⁺⁺ leakage, pore openings, cytochrome c release, etc. will induce cell death. Thus when the intent is to destroy rather than correct hyperproliferating cell only a portion of mitochondria need be compromised. Accordingly, one preferred strategy for triggering death of cancer cells is delivery of a proliferating mitochondrial vector to a targeted cell wherein a sufficient number of the mitochondria are modified either in the mitochondrial genome, mitochondrial membrane, delivery of components to the mitochondria, etc. to cause the mitochondria to elicit cell death.
DNA polymerase is responsible for synthesizing DNA, a key component in the running of biological machinery. Using fluorescence correlation spectroscopy, we demonstrate that the diffusive movement of a molecular complex of DNA template and DNA polymerase enhances during nucleotide incorporation into the growing DNA template. The diffusion coefficient of the complex also shows a strong dependence on its inorganic cofactor, Mg²⁺ ions. When exposed to gradients of either nucleotide or cofactor concentrations, an ensemble of DNA polymerase complex molecules shows collective movement toward regions of higher concentrations. By immobilizing the molecular complex on a patterned gold surface, we demonstrate the fabrication of DNA polymerase-powered fluid pumps. These miniature pumps are capable of transporting fluid and tracer particles in a directional manner with the pumping speed increasing in the presence of the cofactor. The role of DNA polymerase as a micropump opens up avenues for designing miniature fluid pumps using enzymes as engines.
   DNA polymerase as a molecular motor and pump. Sengupta, S. et al, ACS Nano. 2014 Mar 25;8(3):2410-8. doi: 10.1021/nn405963x. Epub 2014 Mar. 6. Abstract.

### Countering Anti-apoptotic Activities

The invention may incorporate actions and/or compositions the impact transcription, translation, cytoskeleton control or other factors that modulate the propensity or ability of proteins which disfavor apoptotic events in the cell. These proteins include, but are not limited to: Bcl2, BcIXI, BcIxES, and Nip3.

### Encouraging Pro-apoptotic Activities

The invention may incorporate actions and/or compositions the impact transcription, translation, cytoskeleton control or other factors that modulate the propensity or ability of proteins which favor apoptotic events in the cell. These proteins include, but are not limited to: Bax, Bak, Bad, Bid, Bim, NoxA, Puma, proline oxidase, p53, cytochrome C, Hsp10, SMAC/DIABLO, apoptosis inducing Factor (AIF), endonuclease G, IAP inhibitor: omi/high temperature requirement protein A2 (HtrA2), adenine nucleotide translocator (ANT), cyclophilin D, peripheral benzodiazepine receptor, and procaspases.

### Mitochondrial and Nuclear Genomes are Distinct but Work in Concert

The mitochondrial genome and the mitochondrion itself have evolved in parallel with the nuclear genome and the cells which mitochondria support. Metabolic processes (the bases of life are divided between these compartments. The mitochondrion is best known for the Electron Transport Chain, the TCA or Krebs cycle for efficient production of ATP. Mitochondria also are responsible for producing acetyl CoA for use in the mitochondrion and cytoplasm. And fatty acid oxidation resides in the mitochondrial matrix.

### Metabolism of Fatty Acids is Shared Between Mitochondrial Matrix and Cytoplasm

Shorter fatty acids can diffuse into the mitochondrion. However, longer fatty acids are reacted with coenzyme A to become esterified as a fatty acyl-CoA. This complex is carried into the intermembrane space, but must be back-converted to acyl- CoA to cross the inner mitochondrial membrane and gain access to the enzymatically active matrix.

Beta oxidation of fatty acids takes a long route. Free fatty acids are carried by a transporter protein e.g., FAT/CD36, SLC27, FATP, and FABPₚₘ from the interstitial space to the cytoplasm. Or fatty acids can be made available internally by autophagy or other degradative processes. In the cytoplasmic compartment the fatty acid is adenylated consuming two active phosphates (ATP→AMP) before a CoA group is added to the fatty acid by fatty acyl-CoA synthase (FACS)to make long-chain fatty acyl-CoA. But long chain fatty acyl-CoAs cannot cross the mitochondrion's outer or inner membranes. Carnitine palmitoyltransferase 1 (CPT1) substitutes carnitine for CoA to form a carnitine-CoA which then crosses the outer membrane to the intermembrane space. This is repeated to cross the inner mitochondrial membrane. The transporter—carnitine translocase (CAT), exchanges long-chain acylcarnitines for carnitine molecules thus recycling carnitine for the next transport. At the inner mitochondrial membrane CPT2 then converts the long-chain acylcarnitine back to long-chain acyl-CoA.

The long-chain acyl-CoA enters the fatty acid β-oxidation pathway that produces one acetyl-CoA from each cycle of fatty acid β-oxidation. In this process each removal of acyl-CoA by acyl-CoA dehydrogenase, yields a shortened fatty acid transenoyl-CoA and one FADH₂. The transenoyl-CoA Is hydrated by enoyl-CoA hydratase to hydroxyacyl-CoA. This is reduced by NAD⁺ and β-hydroxyacyl-CoA dehydrogenase to β-ketoacyl-CoA. Acyl-CoA acetyl-transferase then adds another CoA while cleaving one-acetyl CoA. Acetyl-CoA can condense with oxaloacetate to enter the citric acid cycle as citrate. NADH and FADH₂ produced by both fatty acid β-oxidation and the TCA cycle are used by the electron transport chain to produce ATP.

A partial reverse of this process is used to produce ketone bodies especially essential to the central nervous system when glucose is unavailable. Two acetyl CoAs can be converted by thiolase to acetoacylCoA which HNG-synthase catalyzes to form HMG-CoA. Then HMG-CoA lyase forms one acetoacetate and regenerates a CoA. β-hydroxybutyrate dehydrogenase converts the acetoacetate molecules to β-hydroxybutyrate available to maintain brain activity in the absence of available glucose.

### Krebs Cycle is Normally a Major Mitochondrial Metabolic Emphasis

The Krebs cycle for which the mitochondrion is probably best known is summarized below:

glucose + ATP:

```
      hexokinase
glucose-6-phosphate (G6P) + ADP  → ribose-5-P + NADPH
      [pentose phosphate pathway]
      phosphoglucose isomerase
fructose-6-phosphate + ATP
      phosphofructokinase (PFK) (inhibitors: phosphoenolpyruvate (PEP),
      ADP
fructose-1,6-bisphosphate + ADP
      aldolase
glyceraldehyde-3-phosphate + dihydroxyacetone phosphate
      dihydroxyacetone phosphate
        triose phosphate isomerase
      glyceraldehyde-3-phosphate
(2x) ↓
glyceraldehyde-3-phosphate + Pᵢ + (NAD⁺)
(NAD⁺ → NADH)
      Glyceraldehyde-3-phosphate dehydrogenase
1,3-bisphosphoglycerate + (NADH)
1,3-bisphosphoglycerate + ADP
      phosphoglycerate kinase
3-phosphoglycerate (3PG) + ATP
      phosphoglycerate mutase
2-phosphoglycerate
      enolase [Mg⁺⁺ cofactor]
phosphoenolpyruvate + H₂O [See PFK above]
phosphoenolpyruvate + ADP
      pyruvate kinase
pyruvate + ATP
pyruvate + NADH
      lactate dehydrogenase A (LDH-A)
lactate + NAD⁺
```

### Metabolic Pathways in Blood-Vessel Formation.

The formation of blood vessels depends on the proliferation and migration of endothelial cells—processes that require production of the metabolite acetyl-CoA from mitochondria. Conversion of glucose, glutamine and other nutrients into acetyl-CoA is required for the production of energy and macromolecules, both of which promote endothelial-cell migration to the metabolizing site. The interconnected metabolic pathways make the production of acetyl-CoA, from oxidation of fatty acids, essential for DNA synthesis and endothelial-cell and any other cell proliferation.

Vitamin B3, Niacin: In addition to its well-known redox functions in energy metabolism, niacin, in the form of NAD, participates in a wide variety of ADP-ribosylation reactions. Poly(ADP-ribose) is a negatively charged polymer synthesized, predominantly on nuclear proteins, by at least seven different enzymes. Poly(ADP-ribose) polymerase-1 (PARP-1) is a major participant in polymer syntheses and is important in DNA damage responses, including repair, maintenance of genomic stability, and signaling events for stress responses such as apoptosis. PARP-1 is therefore a prime target when metabolic modulation is in play.

### G Proteins

NAD is also used in the synthesis of mono(ADP-ribose), often on G proteins. Sequencing the human genome has made obvious the number and importance of G proteins for signal transduction, and as targets for therapeutic intervention.

Several G proteins act through stimulating production of cyclic AMP (cAMP) from ATP through stimulating the membrane-associated enzyme adenylate cyclase. cAMP then act as a second messenger that activates protein kinase A (PKA). PKA under different conditions phosphorylates many different downstream targets, including, but not limited to: Anti-Diuretic Hormone (ADH, aka vasopressin), Growth Hormone Releasing Hormone (GHRH), Growth Hormone Inhibiting Hormone (GHIH, aka somatostatin), Corticotropin Releasing Hormone (CRH), Adrenocorticotropic Hormone (ACTH), Thyroid Stimulating Hormone (TSH), Thyrotropin Releasing Hormone (TRH), Lutinizing Hormone (LH), Follicle Stimulating Hormone (FSH), Parathyroid Hormone (PTH), Calcitonin, Glucagon,(human) chorionic gonadotropin ((h)CG), and Epinephrine.

### Vitamin B3—Niacin

NAD and NADP are required for the synthesis of cyclic ADP-ribose and nicotinic acid adenine dinucleotide (NAADP). These compounds control intracellular calcium signaling. Modulating any of these processes has the potential to impair genomic stability which might deregulate cell division and contribute to enhanced cancer activity.

### Vitamin B6—Pyridoxine

High dietary vitamin B6 attenuates and low dietary vitamin B6 increases the risk of cancer. Vitamin B6 is present in many foods so severe deficiency is uncommon. But even in the absence of a clinical deficiency availability of B6 may be sub-optimal, especially with respect to rapidly proliferating cancer cells. B6 is an important enzymatic cofactor. See, e.g., heme synthesis discussed later. Modulating B6 availability to the organism or to a cell or a group of cells in the organism can be one tool in modulating and balancing metabolism in favor of limited proliferation.

### Examples of Key Nodes in Metabolic Control of the Ribose Pathway

NADPH inhibits conversion of G6P to gluconolactone. Ascorbate/Cu⁺⁺, ascorbate/Fe⁺⁺, Cu⁺⁺, diazotetrazole, and GSH are inhibitors of gluconolactonase that converts gluconolactone to 6-phospho-D-gluconate. Triphenylmethane and derivatives: Bromocresol Purple, Bromocresol Purple—salt, Bromochlorophenol blue—salt, Bromophenol blue—salt, Tetraiodophenolsulfonephthalein—salt, ethylenesulfonic acid oligomer, 4-phospho-D-erythronate, 2-deoxy-6-phospho-D-gluconate, 5-phospho-D-ribonate, 6-aminonicotinamide, 6,7-dideoxy-7-phosphono-d-glucoheptonate, 6-deoxy-6-phosphono-d-gluconate, 5-phospho-d-ribonate, 4-Phospho-d-erythronate, each inhibit 6-phosphogluconate dehydrogenase thereby preventing additional NADPH and ribulose phosphate formation. The inhibitory salts are salts most commonly using a monovalent cation, and very often sodium salt is most available in the open market. However, other salts may be selected when the benefits justify the additional efforts required to obtain and deliver them.

Oxythiamine and p-hydroxyphenylpyruvate inhibit transketolase and arabinose-5-phosphate; and fructose 1,6-bisphosphate inhibits transaldolase. This inhibition may in some instances be counterproductive because these enzymes scavenge ribose-5-P and divert the carbon to fructose-6-P and gluceraldehyde-3-P. Ribose-5-P is used for nucleic acid synthesis, but blocking the pathway may have its advantages.

D3 Inhibition of the Hedgehog Signaling Pathway

The hedgehog (Hh) signaling pathway is a developmental pathway which plays a key role in directing growth and tissue patterning during embryonic development. Dysregulation of Hh signaling contributes to the development of a variety of human tumors, including skin, brain, colon, pancreatic, and lung cancers. When constutively activated, this pathway results in the increased expression of Hh target genes, including several forms of the glioma-associated oncogene (Gli) family of signaling proteins. These events are associated with uncontrolled tumor proliferation. Research has demonstrated that the anti-proliferative activity of Hh pathway inhibitors (including, e.g., Cyc, GDC-0449, and VD3) in cultured cancer cell lines does not correlate with pathway inhibition in Hh-dependent cells. However, each of these compounds has modest anti-proliferative effect in multiple cell lines, suggesting either Hh signaling plays a role in preventing cancer cell growth in vitro or that anti-proliferative effects of these compounds are mediated through unidentified cellular mechanisms not associated with Hh inhibition.

With respect to VD3, cellular effects unrelated to Hh signaling likely result from activation of VDR signaling. Therefore, applying the anti-proliferative activity of the VD3 analogues could demonstrate ability to selectively inhibit the Hh pathway.

In several cancer cell lines, increased expression levels of vitamin D metabolizing enzymes for example in U87MG cells suggests that the enhanced anti-proliferative effects may result from the cellular conversion of VD3 to 25-hydroxy-D3 and/or to 1a,25-hydroxy-D3 and to subsequent activation of VDR.

Recent studies have demonstrated that several natural and synthetic cholesterol metabolites, including oxysterols and bile acids, present anti-proliferative effects.

### Cellular Metabolism and Disease

To date, more than 8,700 reactions and 16,000 metabolites are annotated in the Kyoto Encyclopedia of Genes and Genomes (see e.g., http://www.genome.jp/kegg/ pathway.html). At its base, metabolism can be simplified to pathways involving nutrients: carbohydrates, fatty acids and amino acids that are essential for energy homeostasis and synthetic metabolism in humans. In most mammalian cells, growth (anabolic metabolism) occurs only when promoted by extracellular ligands. These growth factors stimulate signal transduction pathways including, but not limited to: the phosphatidylinositol 3-kinase (PI3K)/Akt, and the mammalian target of rapamycin (mTOR) pathway. Many additional pathways and points for modulating same are discussed elsewhere in the description. Activation of PI3K/Akt like other pathways alters the phosphorylation states of numerous target proteins (whose phosphorylation status determines activity levels) which together coordinate cellular activities including those that coordinate cell division or proliferation. But a successful transition from a resting state to growth can only occur if metabolism is adjusted to meet the rising demands for molecules like nucleic acids that are necessary precursors to a cell's proliferation.

Growth factor-induced signaling is a common practice for organisms to coordinate these functions. Underlying this is a requirement for maintaining a bioenergetic state permissive for growth. For a cancer cell to proliferate it must have previously made the macromolecules necessary for both daughter cells and must have consumed and now stored sufficient energy to accomplish the task.

In particular, the PI3K/Akt/mTOR pathway is commonly activated in proliferating cell because it both stimulates a rapid increase in essential nutrient uptake and directs the allocation of these nutrients into catabolic and anabolic pathways needed to produce the energy and macromolecules. Interference with any of these downstream metabolic effects can render the growth factor initial stimulation ineffective.

Dynamic mechanisms also sense cellular energy status and regulate a balance between anabolism and catabolism. Whereas the PI3K/Akt/mTOR pathway promotes anabolism and suppresses catabolism, AMP-activated protein kinase (AMPK) does the reverse. This serine-threonine kinase is a “fuel sensor” that becomes activated during a compromised bioenergetic state such as acute nutrient deprivation or hypoxia. By phosphorylating a number of gatekeeper targets, AMPK down-regulates energy-consuming, growth-promoting pathways like protein and lipid synthesis and up-regulates catabolism of fatty acids and other fuels. This enables the cell to rebalance energy supply with demand.

AMPK also regulates a p53-dependent, cell-cycle checkpoint activated by glucose deprivation thereby limiting growth when glucose supply is weak. AMPK also coordinates expression of stress response genes by migrating to chromatin and phosphorylating histone H2B on its S₃₆. This modulating activity synergizes AMPK's effects on gene expression in the nucleus. As a result, AMPK executes and controls several activities that allow cells to respond emphatically and comprehensively to energy shortage. In mammals, cell growth and proliferation are controlled by extracellular factors that bind to receptors on the plasma membrane that include, but are not limited to: hormones, growth factors, cytokines, specific nutrients, etc. These ligands bind to cell surface receptors and initiate signal transduction cascades that stimulating numerous cellular activities to enable growth and replicative division. Appropriate control of metabolism is required for these effects to achieve valid results. For example, one of the proximal effects of growth factor signaling is to increase surface expression of transporters, for glucose and other nutrients, which when consumed provide energy and metabolic precursors to produce needed macromolecules. Catabolism of these nutrients generally ends with carbon dioxide and energy. If nutrients are present in excess so that flux through these foundational catabolic pathways is satisfied, other pathways branching from core metabolisms are induced to propagate growth signals internally and/or for export.

Hexosamine biosynthesis reinforces growth signals by enabling cells to maintain protein synthesis for example for cell surface expression of growth factor receptors and of nutrient transporters. Acetyl-CoA generated by acetyl-CoA synthetases (ACS) and ATP-citrate lyase (ACL) provides substrate needed to synthesize lipids and other macromolecules and for acetylation reactions that regulate gene expression and resultant enzyme functions. A favorable energy state during growth factor signaling also suppresses AMPK, thereby permitting cells to engage in energy-consuming biosynthetic pathways and to progress through the cell cycle.

The TCA cycle of the mitochondrion serves a biosynthetic role in addition to its more familiar function as energy deliver. Requirements of the rapidly proliferating cells for production of specific biosynthetic products would control the relative importance of the TCA cycle in tumors. Precursors for: protein, lipid, and nucleic acid synthesis are produced in the TCA cycle. Export of these precursors from the cycle to supply macromolecular synthesis is a prominent feature of proliferating cancer cells. Pyruvate carboxylation is one of several mechanisms by which carbon can be resupplied to the TCA cycle to offset precursor export. Such processes, termed anaplerotic pathways, prevent TCA cycle intermediates from becoming detrimentally depleted during cell growth.

18F-fluorodeoxyglucose-PET (FDG-PET), which is commonly used to assess lung tumors, can identify localized areas of intense glucose uptake. Multiparametric MRI would also be particularly useful for this type of analysis, since it can assess regional heterogeneity of perfusion, oxygenation, cellularity, necrosis, temperature, and other characteristics relevant to cancer cell metabolism.

Cells have two ways to produce adenosine triphosphate (ATP) for energy: glycolysis and oxidative phosphorylation (OXPHOS). In glycolysis, glucose is converted to pyruvate, while generating NADH from NAD⁺ and ATP from ADP and inorganic phosphate. If the pyruvate is reduced to lactate, NAD⁺ is regenerated and glycolysis continues. Although glycolysis is rapid, it is deemed inefficient because most of the energy that could be generated from glucose is lost when the cell secretes lactate, a three carbon molecule retaining significant energy in its bonding structure. In contrast, OXPHOS is highly efficient about 20-fold more efficient per ATP molecule generated. When substrates like pyruvate are oxidized in the mitochondria, reducing equivalents are provided to the electron transport chain, creating a proton gradient that drives ATP synthesis. The vast majority of cancer cells use both glycolysis and OXPHOS together to satisfy metabolic needs, although OXPHOS I reduced in its importance as mitochondria are co-opted for supporting essential proliferation pathways, the balance between the two can vary widely in different cancer types and at different phases of cancer development.

Genes involved in the rebalancing relate to a large number of the cells' pathways and their enzymes, including, but not limited to: STAT1, Akt, Jak/Tyk2, CUG2, HGPRT, SETDB1, LDH1, etc.

Especially notable or pathways leading to lactic acid formation, thus sparing mitochondrial activity from having to metabolize pyruvate, pathways leading to purine and pyrimidine manufacture to support nucleic acid synthesis, pathways leading to angiogenesis. pathways sparing the proliferating cell from cell death or apoptosis, pathways that may be activated to drive the proliferating cell towards cell death or apoptosis and pathways that control cell division and cell cycle.

A low-molecular-weight compound secreted appears responsible for enhanced CLL cell survival. This compound is probably the amino acid cysteine, one of the three amino acids required to synthesize glutathione, a protector against oxidative damage and maintenance of volume in tissues such as the cornea. Most cells, including cancer cells, do not rely on extracellular cysteine for their glutathione biosynthesis. Rather, cells take up the more abundant and more stable oxidized form, cystine (two cysteine molecules joined by S—S bonding). Cystine is readily reduced to cysteine for synthesizing polypeptides inside the cell. Cystine is taken up through the Xcantiporter, a multimeric amino-acid transporter that exchanges glutamate for cystine at the cell surface.

Hypoxic tumor cells appear to favor conversion of glucose to lactate, which is disposed of by secretion (or export) into the interstitial fluid (extracellular compartment) where it can be metabolized by cells in areas of more abundant oxygen—either because of better vascularization of because of lower metabolic demands. In breast cancer, tumor cells derived from the luminal epithelium synthesize glutamine de novo before secreting it. So while controlling glutamine availability may be an important support in methods of the present invention the same method(s) will not apply to all instances where the invention is practiced. Obviously, these luminal epithelial derived cancer cells and other cancer cells with similar metabolic modifications can thrive under conditions of glutamine deprivation.

By contrast, cells derived from the basal epithelium do not synthesize glutamine and therefore require an extracellular source. These cells can be rescued by co-culturing them with glutamine-secreting luminal cells, raising the possibility of regional heterogeneity in glutamine dependence in normal and tumor- tissue.

One mechanism evidencing benefits of controlling glutamine access is provided by understanding the mitochondrial enzyme glutaminase C (GAC) which catalyzes the hydrolysis of glutamine to glutamate plus ammonia (NH₄⁺ ). Such glutamine focused diversion appears to be a key step in the modifications of metabolism, in general, and of glutamine, in particular, by cancer cells. Because glutamine is necessary for a range of biochemical reactions, notably including nucleotide and protein synthesis, glutamine analogs like the GLS1 inhibitor diazo-O-norleucine (DON) may not be ideal candidates for cancer drugs as a broad class.

However, two classes of allosteric GAC inhibitors have been identified and may present more promise as active ingredient compounds for cancer therapeutics. One of these inhibitor groups consists of analogs of bis-2-(5-phenylacetamido-1,2,4-thiadiazol-2-yl)ethyl sulfide (BPTES), a reversible GAC inhibitor. X-ray crystal structures of the GAC-BPTES complex show that BPTES effectively traps GAC as an inactive tetramer. A second, more recently identified, class of allosteric GAC inhibitors, a class that is highly specific for inhibiting cancer cell growth while having little effect on normal (nontransformed) cells is represented by the benzophenanthridinone 968. Oncogenic Dbl induction did not cause marked increases in glucose-fueled anaplerosis, as measured by ¹³C enrichment in citrate, when using [U-¹³C]-glucose as a tracer, demonstrating that a highly specific stimulation of glutamine metabolism accompanies Rho GTPase-dependent transformation.

Reductive Carboxylation Supports Growth in Tumor Cells with Defective Mitochondria

Mitochondrial metabolism provides precursors for macromolecules in growing cancer cells. In normally functioning tumor cell mitochondria, the oxidative metabolism of glucose-derived and glutamine-derived carbon produces citrate and acetyl-coenzyme A for lipid synthesis, an important activity to support tumorigenesis. And some tumors bear mutations in the citric acid cycle (CAC) or electron transport chain (ETC) that disable normal oxidative mitochondrial function. Yet in a large number of tumors, the citric acid cycle and the electron transport chain remain functionally intact, though de-emphasized in activity. It is not understood how cells from CAC and/or ETC deficient tumors generate precursors necessary for macromolecular synthesin support of proliferation. But cells with defective mitochondria likely use glutamine-dependent reductive carboxylation rather than oxidative metabolism as the major pathway of citrate formation. This pathway uses mitochondrial and cytosolic isoforms of NADP1/NADPH-dependent isocitrate dehydrogenase, and subsequent metabolism of glutamine-derived citrate provides both the acetylcoenzyme A for lipid synthesis and the four-carbon intermediates needed to produce the remaining CAC metabolites and related macromolecular precursors. Cells with intact CAC and ETC may down-regulate these paths to better support the synthesis of purines, pyrimidines and other macromolecule precursors. This is probably a strong hypothesis given that reductive, glutamine-dependent pathway is the dominant mode of metabolism in rapidly growing malignant cells containing mutations in complex I or complex III of the ETC, in patient-derived renal carcinoma cells with mutations in fumarate hydratase, and in cells with normal mitochondria subjected to pharmacological ETC inhibition.

Induction of a versatile glutamine-dependent pathway that reverses many of the reactions of CAC supports tumor cell growth, and illustrates how cells may generate satisfactory pools of CAC intermediates in the face of impaired mitochondrial metabolism.

As expected for cells with defective oxidative phosphorylation, a model cell line, CYTB 143B cells, had higher glucose consumption and lactate production than WT143B cells, demonstrating the metabolic shift towards aerobic glycolysis.

### Glutamine and Cancer: Cell Biology, Physiology, Clinical Strategies

In mitochondria, under the influence of Hif1α, pyruvate dehydrogenase kinase (PDH), (PDK) blocks the activation of mitochondrial pyruvate dehydrogenase thereby limiting the pyruvate conversion into acetyl-CoA. Hif1α (hypoxia inducible factor 1α) also stimulates expression of LDH-A to generate NAD⁺ . LDH-A hyperactivity appears essential for scavenging pyruvate to maintain NAD⁺ and/or to remove pyruvate stimulus of the mitochondrial pyruvate to acetyl-Co-A Krebs mission. But Krebs is still able to partially cycle when glutamine is deaminated to glutamate in a reaction supporting synthesis of the pyrimidines and purines used for nucleic acids. The glutamate enters the mitochondrion as α-ketoglutarate which progresses through maleate, exits the mitochondrion then is converted to pyruvate and lactate.

Protein kinase B (PKB, aka Akt) is important for regulating the glycolytic over OXPHOS favoritism. PKB/Akt after being phosphorylated by phosphatidylinositol 3 kinase (P13K) takes residence in the plasma membrane and inhibits or slows several paths such as increased cAMP response element binding protein (CREB) with actions impacting Alzheimer's, spatial and long term memory, c-fos, tyrosine hydroxylase, time keeper genes—Periodl and Period2 (PER 1 and 2), and many other important neuropeptides such as somatostatin. The target of CREB is the sequence TGACGTCA which will be left unhindered when it benefits from C methylation.

CREB also influences the plasma membrane though its activation of P13K which controls positioning and polarity of receptors in plasma membranes. P13K activation is essential in forestalling differentiation in favor of proliferation and thereby plays a key role in supporting cancer proliferation and slowing apoptosis. Stimulation by insulin, insulin-like growth factor 1 (ILGF1 or an alternate name somatomedin C), calmodulin, epidermal growth factor, sonic hedge-hog, and the like, favors “proliferation and growth”' over functional differentiation and culling (apoptosis).

Homeobox 9 (HB9), phosphatidylinositol-3,4,5-trisphosphate 3-phosphatase (PTEN), Glycogen Synthase Kinase 3 Beta (GSK3B)

The diversion of G6P to the R5P pathway, to generate ribose-5-phosphate and NADPH, supports the enhanced nucleotide synthesis necessary for transcription and translation. The NADPH is also a major anti-oxidant and participant in bio-synthesis. The 3PG is a reactant for both amino acid and fatty acid and other synthesis.

3PG is notable for its conversion to serine which serves as a carbon source for the folate cycle through its conversion of tetrahydrofolate (THF) to methyl-tetetrahydrofolate (mTHF). NADPH is oxidized as part of the folate cycle. Monocarboxylate transporter 4 (MCT4) is necessary for removing the lactate from cell's cytoplasmic space. Since the lactate production is enhanced in proliferating cancer cells, especially growing, vascularization deprived, and/or hypoxic cancer cells, interference with formation of intact functioning transporter including, but not limited to stopping or altering: transcription, translation, expressing, processing, transport to or insertion in plasma membrane and maintenance within the membrane will seriously compromise cell survival.

A seemingly opposite strategy can augment or synergize this result. Since neighboring cells, especially neighboring well-oxygenated cancer cells, may remove lactate from interstitial space and cycle it though lactate dehydrogenase for metabolic use or may otherwise remove lactate, by blocking or slowing MCT1, the relevant lactate uptake transporter, a toxic buildup of lactate in the interstitial space which then would back up into cells to can promote necrosis or apoptosis of these cells.

In some tumor situations, blocking lactate uptake can severely increase the demand for glucose, which when unmet starves the cells into a necrotic or apoptotic, or extreme quiescent state. Glycine also is involved in the folate cycle. Accordingly, stopping glycine C-transferase activity and/or glycine dehydrogenase activity phosphoaminotransferase (PSAT) and/or serinehydroxymethlase (SHMT) should take away these pathways for tumorigenesis. Especially in colon cancers stimulating fragile histidine triad may maintain genome stability, but its absence is compatible with rapid mutation as observed in cancer cells.

Activation of the PI3K-Akt pathway will increase glucose uptake and metabolism because Akt phosphorylates and inactivates FOXO. This down regulates PGC1α and inhibits mitochondrial biogenesis. When MYC is activated glutaminolysis is induced—glutamine is converted to α-ketoglutarate (αKG). Then reductive carboxylation of αKG, using NADPH-linked IDH2, results in isocitrate and more citrate available for export to the cytosol, where isocitrate is available for conversion back to αKG by NADP⁺-linked IDH1. Or citrate may be exported from the mitochondrial matrix to the cytosol where it is cleaved by ATP citrate lyase (ACL) to produce oxaloacetate (OAA) and acetyl-CoA.

### Diverse Functions of Glutamine in Metabolism, Cell Biology and Cancer

Glutamine is an amino acid, one of the constituents of proteins. Glutamine is also an acceptable substitute for glucose as the cell's fuel. The ready alternatives available as substrate for various metabolic functions, and alternative pathways available to achieve the necessary functions, suggests two main approaches for external control of unwanted cell growth and proliferation. A first approach would be to block metabolism at an initiation step critical to many downstream paths or to block a junction point critical to several alternative path. A second approach would be to therapeutically manipulate several interfacing or parallel paths. Glutamine because it can participate in many functions, including, but not limited to: a carbon source for building biomolecules, an energy source for generating needed ATP, and a conduit of nitrogen between cells and parts of cells. Glutamine with all its use is not surprisingly the most common amino acid (about ⅕ of the amino acids) free in circulating blood. Glutamine although capable of being synthesized in mammalian cells, often is in short supply for all the metabolic demands it can satisfy. Glutamine is exported to circulation as a non-toxic carrier of NH₄⁺ for example from breakdown of other amino acids. Glutamine is a major source of urea, the chemical carrier of nitrogen out of the body in renal waste. As a nutrient for cancer cells glutamine is often, but not always, available from the circulatory system. Another prime source of glutamine is proteins as they are recycled during normal metabolic processes. The extraordinary consumption of glutamine in cancer cells is evident in the activity of oncogenic RAS to stimulate macropinocytosis, a process through which extracellular molecules, e.g., proteins are ingested by the cell in the form of macropinosome vesicles. These vesicles can merge to intracellular lysosomes for degradation of the engulfed proteins to useful building blocks. Amplifying this macropinosomic lysosomic activity by internal or external signaling paths, like amplifying other lysosomic activities is one means of initiating apoptotic cell death. Mitochondria participate in glutamine recycling through several aminotransferases discussed below. A glutamine transport protein e.g., SLC1A5 internalizes circulating glutamine. In the cytoplasm, glutamine can be converted to nucleotides and uridine diphosphate N-acetylglucosamine (UDP-GlcNAc). Nucleotides are essential molecules for making genes in dividing cells.

N-glycosylation serves to stabilize proteins by maintaining appropriate 3D folded structure and to package for secretion to extracellular space. Alternatively, glutamine can be converted to glutamate by glutaminase (GLS or GLS2). The glutamate may be used to generate glutathione (an anti-oxidant protectant) or may be processed into other metabolic substrates, such as a-ketoglutarate (a-KG). The importance of this path is emphasized by the parallel pathways, i.) glutamate dehydrogenase GLUD which comes in two forms, GLUD1 and GLUD2, and ii.) aminotransferases. GLUD is activated by ADP and inhibited by GTP, palmitoyl-CoA and SIRT4-dependent ADP ribosylation. Leucine by itelf allosterically activates GLUD and by acting through mTOR suppresses SIRT4 expression thereby accentuating GLUD activity even more. When ADP levels increase e.g., by consumption of ATP in excess of creation, this may operate as a signal for GLUD to increase its ATP output. GLUD has NH₄⁺ as a product which might be detoxified by conversion to glutamine! Whereas the aminotransferase path is used to make other amino acids. Aminotransferase reactions can occur both in the mitochondria and in the cytoplasm. In some tumors 50% or more of the non-essential amino acids used to build proteins are derived from glutamine. And glutamine through its involvement in aspartate synthesis is a key element for making the purines and pyrimidines necessary for nucleic acids. Then in the mitochondrion α-KG can participate in the tricarboxylic acid (TCA) cycle through succinate and fumarate to malate thereby providing ATP for the cell.

Malate can leave the TCA cycle to produce pyruvate and NADPH. When it remains in the mitochondrion malate cycles to oxaloacetate (OAA) which may leave the cycle as aspartate to support nucleotide synthesis, e.g., DNA or tRNA for a dividing or rapidly metabolizing cell. As another option available in the cell's metabolism, α-KG can reverse through the TCA cycle, in a process called reductive carboxylation (RC) to form citrate, to make acetyl-CoA and lipids. The requirements of tRNA (and probably to a lesser degree, mRNA) and DNA for the growing and proliferating cell are perhaps the most likely rational for a cancer cell's metabolic shift in favor of glutamine. Two glutaminase enzymes (GLS and GLS2) are differentially expressed depending on tissue type. GLS which has two alternative splice forms (GLC and KGA)is activated by phosphorylation, but receives feedback inhibition by its glutamate product. GLS2 however increases activity as its NH₄⁺ increases abundance. These enzymes are regulated by sirtuin 5 (SIRTS) which down-regulates GLS and SIRT3 which up-regulates GLS2 (especially during times where caloric intake is wanting). pH is an important modulator of GLS mRNA and its expression and activity can be controlled at the site of transcription (in the nucleus), and later in cytoplasmic environment by microRNAs and RNA binding proteins directing mRNA processing and alternative splicing. Splice variant GAC appears more prevalent in many cancers and is the more active variant. The cell's favoring of this variant would not be apparent in a nuclear genome sequencing, but might be seen in a complete sequence analysis that also monitors expression. GLS2 can be turned off by methylation which has been observed in some cancers, especially hepatic forms. GLS2 methylation may also be important for cancer cell creation in that this enzyme may have another quality or side effect in its propensity to bind RAC1 cutting metastasis.

The aminotransferase family includes several forms. Better characterized family members include alanine aminotransferase (aka glutamate—pyruvate transaminase), aspartate aminotransferase and phosphoserine aminotransferase (PSAT1). Alanine aminotransferase comes in a mitochondrial isoform GPT2 and a cytoplasmic isoform, GPT. Similarly, aspartate aminotransferase has a cytoplasmic isoform, GOT1 and a mitochondrial isoform, GOT2. PAT1 appears to be preferentially expressed in tumor cells and thus controlling its activity can be one tool for stressing cancer cells. In cancer cells where hypoxia-inducible-factor-α (Hifα) is constitutively expressed or where mitochondrial participation in fatty acid synthesis is severely compromised, glutamine may see further use in reductive carboxylation to synthesize fats.

Glutamine metabolism is crucial for cellular reactive oxygen species (ROS) homeostasis. Glutathione (GSG), one of the important reactive oxygen scavengers, requires glutamine as a raw material for the amino acid components of GSG. Many studies have shown that glutamine availability is rate limiting in GSG synthesis. ROS effects are complicated. Under some conditions increased ROS (a sign of cell stress) initiates apoptosis. But some cancers as part of their development process have survived by downplaying the apoptotic input of increased ROS. In these cases, ROS can cause internal oxidative damage within the cells. Glutamine also is involved in the TOR pathway. TOR encourages growth and inhibits autophagy. Glutamine suppresses pro-apoptotic action of GCN2 and Integrated Stress Response (ISR).

Oncogenic genes upregulate glutamine uptake and metabolism as observed in the Q (glutamine) metabolism stimulated by HIF2 and MYC. When glutamine is metabolized, its carbon mass is preserved chiefly in amino acids and fats while the nitrogen is an integral component for nucleic acid synthesis. Through aspartate transamination glutamine can also contribute carbon atoms to purines and pyrimidines of the nucleic acids. Glutamine can serve an intracellular signal through mTOR to activate carbamoyl-phosphate synthetase 2, aspartate transcarbamylase, and dihydroorotase (CAD), responsible for using nitrogen from glutamine to synthesize pyrimidine. Although tumor cells tend to have large intracellular pools of glutamate, maintaining these pools rests on the ability to convert glutamine into glutamate because glutamine is an abundant extracellular nutrient and glutamate is not. This process is largely because of the activity of phosphate-dependent glutaminase (GLS), a mitochondrial enzyme that is highly expressed in tumors and tumor cell lines.

Classical experiments have shown that GLS activity correlates with tumor growth rates in vivo, (Knox et al. 1969; Linder-Horowitz et al., 1969), and experimental models to limit GLS activity resulted in decreased growth rates of tumor cells and xenografts.

The rate-limiting step in the formation of hexosamine is catalyzed by glutamine:fructose-6-phosphate amidotransferase, which transfers glutamine's amido group to fructose-6-phosphate to form glucosamine-6-phosphate, a precursor for N-linked and O-linked glycosylation reactions. These reactions are necessary to modify proteins and lipids for their participation in signal transduction, trafficking/secretion and other processes. Impairment of glucosamine-6-phosphate production is thus expected to reduce cell growth and to interfere with cell signaling. Surprisingly, glutamine:fructose-6-phosphate amidotransferase activity can be suppressed by expressing an antisense GLS complementary DNA in some breast cancer cells. The disturbances of 0-linked glycosylation pathways, alters glycosylation of the transcription factor Sp-1 and increases its transcriptional activity. Glutathione (GSH) is the major thiol-containing endogenous antioxidant and serves as a redox buffer against various sources of oxidative stress. In tumors, maintaining a supply of GSH is critical for cell survival because it allows cells to resist the oxidative stress associated with rapid metabolism.

GSH is a tripeptide of glutamate, cysteine and glycine and its formation is highly dependent on glutamine. Not only does glutamine metabolism produce glutamate, but the glutamate, pool is also necessary for cells to acquire cysteine, the frequent limiting reagent for GSH production. Glutaminase activity generates free ammonia, a potentially toxic metabolite. Without a mechanism to dispose of ammonia rapidly, intracellular ammonia concentrations would reach several hundred mmol/l within a few hours which would be expectedly toxic to most cells in the area. It is not understood how tumor cells dispose of ammonia during rapid glutamine catabolism. The traditional view held that passive diffusion of the gaseous form (NH₃) across the lipid bilayer accounted for essentially all ammonia transport. This simple model does not provide adequate explanation for some tissues with a high demand for ammonia transport. For example, in the kidney, in which ammonia metabolism is a key mediator of acid-base homeostasis, a number of protein transporters exist to traffic ammonia, as NH₃ and/or NH₄. These systems include ion channels, aquaporins and Rh glycoproteins, some of which are overexpressed in tumors. Although the exact mechanism of tumor cell ammonia secretion has not been proven, the process bears therapeutic potential. Blocking ammonia secretion would, presumably either suppress net glutamine consumption or cause toxic intracellular accumulation of ammonia, both of which should impair cell survival and growth.

Other reports have identified a role for glutamine in extracellular signal-regulated protein kinase (ERK) signaling pathways. This phenomenon has been best characterized in intestinal epithelial cells, which consume glutamine as their major bioenergetic substrate and require glutamine for both proliferation and survival. Addition of glutamine is adequate for stimulating ERK signaling within a few minutes in porcine intestinal epithelial cells, and it enhanced ³H-thymidine incorporation. In rat intestinal epithelial cells, glutamine was shown to be comparable to serum in preventing apoptosis, and it stimulated a sustained activation of ERK signaling. The importance of glutamine as a supporter of tumorigenic activity should not be downplayed.

Inhibitors of the ERK pathway have eliminated the protective effect of glutamine supplementation. It was not clear from these studies whether glutamine import alone was required for the effects, or whether the cells needed to metabolize glutamine to activate ERK signaling. Consistent with glutamine's effects on cell signaling, a number of reports have shown that it also influences gene expression. In cell lines, addition of glutamine increases expression of the pro-proliferation factors c-jun and c-myc within a few minutes and promotes cell survival through the negative effects on growth-inhibitory and pro-apoptotic factors such as CHOP, GADD45, Fas and ATF5. In Ehrich ascites tumor cells, GLS knockdown led to enhanced phosphorylation, DNA binding and transcriptional activity of Sp1. In HepG2 hepatoma cells, glutamine was required for the induction of manganese superoxide dismutase expression that accompanied the depletion of essential amino acids.

Glutamine's involvement in manganese superoxide dismutase expression was blocked by inhibiting the TCA cycle, ERK1/2 or mTOR, suggesting that an integration between mitochondrial glutamine metabolism and signal transduction facilitates the effect.

Evidence shows that glutamine also modulates immune responses, though it is unclear exactly through which mechanistic paths these changes are achieved. Conceivably, glutamine could exert its effects through redox homeostasis, bioenergetics, nitrogen balance or other functions. During radiation-induced oxidative stress in the rat abdomen, pre-treatment of the animals with glutamine significantly decreased tissue inflammation and expression of nuclear factor-kB. Glutamine may be available to buffer the redox cell's capacity.

Nuclear factor-kB likely is a key mediator that links glutamine availability with stress responses, since there is an inverse correlation between glutamine abundance and nuclear factor-kB-mediated gene expression.

The role of glutamine as an immunomodulator in cancer but appears promising in that the avid consumption of glutamine by tumors reduces glutamine availability for neighboring cells, and can modulate local nuclear factor-kB signaling and expression of inflammatory mediators in the stroma. Because tumor cells are exposed to many nutrients simultaneously, achieving a comprehensive view of tumor metabolism requires an understanding of how cells relate these pathways into an over-arching metabolic phenotype. For different tumor cell types and for different tumors pathway emphases would most likely vary. It is expected that the skilled artisan in practicing this invention to its best advantages would investigate glutamine effects, either by assay or trial and error or a combination thereof.

Consequently, considerations relating to glutamine should not ignore the rapid glucose utilization that often accompanies cell proliferation. The rates of glucose and glutamine consumption in general far outpace the utilization of other nutrients available to the cell. Presumably, this modified metabolism supports both bioenergetics and the production of biomacromolecule precursor pools while sparing other energy-rich substrates, such as fatty, acids and essential amino acids, for their direct incorporation into the biomacromolecules. Increased glucose breakdown provides building blocks for the synthesis of nucleotides (via glucosamine and the pentose phosphate pathway) and amino and fatty acids (from intermediates formed in the glycolytic and tricarboxylic acid cycles). In addition, local acidification of the tumor microenvironment may facilitate tumor invasion. The enhanced activity of the pentose phosphate shunt may lead to an elevated production of NADPH and glutathione (GSH) (which would increase the resistance of tumor cells against oxidative insults and some chemotherapeutic agents).

Heme Synthesis—Cooperation between Mitochondrial Matrix and the Cytoplasm

Heme, an iron chelate protein, exemplifies cooperation between cytoplasmic and mitochondrial metabolisms. Heme proteins are found in all cells. The heme group I common where oxygen is found. While probably most known for the heme presence in hemoglobin and myoglobin. Heme is also a component of metaloproteins such as cytochromes, including, but not limited to: p450, b-245, c, d, f, etc. Catalase is an important protector inside most cells from ROS damage, e.g., from hydrogen peroxide (H₂O₂). Catalase sports four heme groups.

Thus, heme synthesis is an important component of cell's ROS defenses. And modulation of heme synthesis (several suggestions below) is a tool available for stimulating necrosis and/or apoptosis.

Heme synthesis is started in the mitochondrion where glycine, brought into the mitochondrial matrix by SLC25A and succinyl-CoA, react to form α-amino-β-ketoadipate in the presence of pyridoxal phosphate (vitamin B6) as a cofactor for the d-aminolevulinate synthase (ALAS) enzyme which then decarboxylates the complex to form d aminolevulinic acid (ALA). CLPX acts as a chaperone to coordinate association of B6 with ALAS thus stabilizing and activating the complex.

Nutritional deficiency of vitamin B6 can limit this reaction and thus heme synthesis. The d-aminolevulinate synthase enzyme is not constitutively expressed and has a short half-life. Expression of the enzyme is induced in the presence of barbiturates and steroids such as testosterone and oral contraceptives that sport a 4,5 double bond that is accessible to 5-β-reductase which itself is induced during puberty. Expression of d-aminolevulinate synthase is inhibited by negative feedback from heme and by hematin.

ALA then is transported to the cytoplasm where d aminolevulinic acid hydratase (aka porphobilinogen synthase) condenses two ALA molecules to synthesize porphobilinogen. Zn⁺⁺ is a cofactor for this enzyme. But Pb⁺⁺ has high affinity and can displace Zn⁺⁺ and inactivate this enzyme.

Lead poisoning effect on this enzyme results in increased ALA in cells and blood. Since ALA cannot progress to eventual heme synthesis there is no heme feedback to suppress ALA synthesis. ALA is a neurotoxin possibly because of the ROS it creates and possibly because it mimics the neurotransmitter, γ-aminobutyric acid.

Four porphobilinogen molecules are condensed by uroporphyrinogen I synthase to form a linear tetrapyrrole which can isomerize non-enzymatically into uroporphyrinogen I or enzymatically with uroporphyrinogen III cosynthase into uroporphyrinogen III. Uroporphyrinogen III is a substrate for vitamin B12 synthesis and chlorophyll synthesis as a branch off this heme synthesis pathway.

Uroporphyrinogen decarboxylase decarboxylates acetic groups of both uroporphyrinogen I and uroporphyrinogen III changing these groups to methyl groups and forming coproporphyrinogen I and coproporphyrinogen III, respectively. The fate of coproporphyrinogen I in the cell is unknown and may be a dead end synthetic product.

Coproporphyrinogen III then migrates back into a mitochondrion through an ATP dependent carrier ABCB6 and is oxidized by coproporphyrinogen III oxidase to form protoporphyrinogen IX.

Protoporphyrinogen IX oxidase aromatizes the ring by converting methylene bridges of protoporphyrinogen IX to methenyl bridges in protoporyrin IX. The resonance bonding improves stability of the molecule.

Ferrochelatase(FECH) then adds Fe⁺⁺ to protoporphyrin IX while reducing ascorbic acid (vitamin C) and cysteine and releasing two H. Lead which inhibited ALA also inhibits ferrochelatase.

Iron is made available to FECH in the mitochondria though a transmembrane carrier, SLC25A37 stabilized with ABCB10 bound to FECH. Then finally the HEME is exported to the nucleus through FLVCR1b for cytosolic incorporation of heme into metaloproteins.

While the Organism Cannot Survive without Surviving Cells, Survival of Each Cell May Not Optimize Survival of the Organism

Cancer cells often upregulate the rate-limiting processes and enzymes of glycolysis, including glucose transporters, for instance as a result of the constitutive signaling through the Akt pathway or as a result of the expression of oncogenes including Ras, Src or Bcl-Abl. Failure to adapt these behaviors would be incompatible with the cell's survival. So only cells effectively navigating these changes will survive to be observed. But since all living things in their creation have a built in drive to survive, when cell's begin to be stressed in a cancer leaning direction, the cell's evolved defense will kick in to preserve life of the cell but may not support survival strategies of the organism.

Cancer cells can accumulate defects in the mitochondrial genome, leading to deficient mitochondrial respiration and ATP generation. In some cases, mitochondrial germline mutations have been shown to provide a genetic predisposition to cancer development. This would be expected because all metabolic defects or changes can be expected to stimulate compensatory reactions which will induce further compensations, etc., within the cell.

In most cases, however, historically, such mutations are acquired during or after oncogenesis. It appears that acquired mutations in mitochondrial DNA fall into two classes. A first category includes severe mutations that inhibit oxidative phosphorylation, increase the production of reactive oxygen species (ROS) and promote tumor cell proliferation. Milder mutations could permit tumors to adapt to new microenvironments, especially when tumors progress and metastasize.

Cancer cells may adapt to decreased oxygen tension (hypoxia) that is characteristic of most, if not all solid tumors as the pre-malignant lesion grows progressively further from the blood supply. In this case, the adaptation to hypoxia would be to durably shut down mitochondrial respiration and to switch on glycolytic metabolism.

In luckily specific cases, mitochondrial enzymes can act as tumor-suppressor proteins whose mutation indirectly engenders aerobic glycolysis. The inactivating mutation of mitochondrion-specific proteins such as succinate dehydrogenase (SDH subunits B, C or D) and fumarate dehydrogenase is an oncogenic event, causing phaeochromocytoma (in the case of SDH mutations) and leiomyoma, leiomyosarcoma or renal carcinoma (in the case of fumarate dehydrogenase mutations). The loss of function of succinate or fumarate deyhdrogenases results in the accumulation of fumarate and succinate in the cytosol, respectively. This, in turn, favors the activation of the transcription factor hypoxia-inducible factor (HIF) and generates a pseudohypoxic state accompanied by HIF-dependent reprogramming of the metabolism towards aerobic glycolysis.

It is conceivable to inhibit glycolysis either by targeting glycolytic enzymes or by attempting to release hexokinase from its mitochondrial receptor, VD). Inhibitors of glycolytic enzymes that have been successfully used to slow down the growth in human tumors transplanted to mice include 3-bromopyruvate (an inhibitor of hexokinase) and oxythiamine (an inhibitor of the transketolase-like enzyme).

Some glycolytic inhibitors are already being evaluated in clinical trials. This applies to 2-deoxyglucose (an inhibitor of the initial steps of glycolysis) as well as to lonidamine (TH-070), an inhibitor of glycolysis that also has direct pro-apoptotic properties.

IL-2 amplifies the body's immune system, while the TGF-β-inhibitor lessens the cancer cells' ability to evade the immune system. Nano devices underdevelopment might be used to administer these or other therapeutic compounds to relevant (diseased) locations. These novel nano devices, mentioned but not required to practice the present invention can in “smart” form be outfitted with sensors and brakes for attachment or movement stoppage to at that location deliver the ported therapeutic or they may remain as marker targets for a second porter to deliver one or more therapeutics to the relevant site.

In an especially elegant version, these nanosensors are equipped with simple diagnostic tools and can be queried to report efficacy of any treatments in their vicinity.

A nanogel delivery system can be used for multiple therapeutics or therapeutic combinations.

### Controlling Proliferation

The cell cycle consists of a state of quiescence (G₀), a first gap phase (G₁), the DNA synthesis (S phase) a second gap phase (G₂), then mitosis (M), the actual cell division phase. Retinablastoma protein phosphorylation by a CDK/cyclin complex allows release of transcription factor E2F that can activate several genes including, but not limited to: cyclins A, D and E. CIP/KIP family members p21CIP1, p27KIP1 and p57KIP2 assist CDK/cyclin association. p53 regulates p21CIP1. p16INK4a and p14ARF are tumor suppressors (encoded by the same gene in overlapping reading frames)!! p16INK4a is inactivated in many cancers. p14ARF can maintain cycle arrest in G₁ or G₂. It complexes with MDM2 to prevent MDM2 from neutralizing p53 thereby transcriptionally activating cyclin-dependent kinase inhibitor 1A or may induce apoptosis. Hyperexpression of cyclins is one hallmark of cancer.

All patents and patent applications referenced herein are hereby in their entireties incorporated by reference.

### Illustrative Examples

As a general process a cell or zone of cells presenting abnormal metabolism is identified. Cells manifesting only initial tendency towards hyperproliferation and/or cancer may be treated and directed back to mainstream metabolism. However, in extreme metabolic digressions, one or more cells may be directed to follow a normal systemic process of cell death.

For example, aberrant metabolism may be detected by one or more physical and/or chemical metabolic indicators such as a local temperature increase from the cellular or mitochondrial chemical activity and/or excess hydrogen ion (H⁺ ) production (resulting in a lowered pH). For increased specificity in identifying the cells progressing along a hyperproliferative or other hypermetabolic path, using a plurality of signals should reduce off-target effects.

In essence chemical and/or physical signals are detected and used to target and correct altered metabolisms. The probe(s) would test whether a cell or zone presented an increased temperature (one indicator of excess or elevated metabolic activity). This probe may be sensitive to another factor such as a chemical presence. E.g., the probe may only bind a certain ligand at elevated temperature, may be activated by increased ion concentration—such as H⁺, and/or may be CO₂ or O₂ dependent.

So a probe or set of probes will test for factor A, temperature in this example, and then factor b, here pH. When both conditions exist, the probe(s) may become activated to steer corrective metabolic events or to eliminate cells that have progressed beyond corrective capabilities. The dual sensor probe may operate as a beacon merely signaling cells requiring return towards normal metabolism. This probe may then serve as a ligand or activator of another compound, a collector of energy, such as electromagnetic radiation and/or as a blocker preventing another molecule from supplying or further activating metabolism at the target cell.

As an example, one probe, e.g., a nanosensor probe, may be both pH and temperature sensitive, concentrating in or on a cell manifesting an above threshold temperature or a temperature exceeding those of near tissues. The temperature sensation and effect may be pH sensitive where, e.g., a lower temperature difference is flagged as pH decreases, may be activated to bind and/or to become active only below a pH threshold. The probe may distribute across a pH gradient favoring distribution/compartmentalization where H⁺ is higher.

Another example makes use of a plurality of probes. For example, sensor T may distribute according to temperature and sensor H may distribute in accordance with pH. Where concentrations of both are elevated they may interact for intended effect.

Other interactions of two or more sensor probes which attract active moieties to the intended target cell or zone of cells are possible. Active sensors or moieties may exert activity through binding an intended cell receptor, through enzymatic action, through scavenging substrate or metabolite, through inducing or inhibiting protein expression, though affecting intercellular binding, communication, through recruiting or activating natural body substances or components, etc.

Vesicles, sensitive to heat, pH, ROS or other chemical attractant or binding agent may serve as couriers for one or more effector molecules. Engineered viruses may be activated at the targeted site, for example through binding to one or more probes, and exert desired outcome(s). Carrier protein, lipids or carbohydrate molecules or combinations thereof may stabilize probe or effector molecules during transport and/or delivery.

A vesicle whose lysis is exacerbated by higher temperatures acidic conditions, or both, may serve to deliver membrane binding agents to areas of lysis. Such binding agents may be inhibitors or ligands for any one or more cell surface markers, e.g., a transport protein, a receptor protein, an adhesion protein, etc., but since their availability for these agents to bind would follow lysis of the temperature and/or pH sensitive vesicle, these agents would be restricted to acting in the relevant zones of pH and/or temperature and, perhaps in some embodiments, one or more additional hypermetabolic harbinger(s).

In the United States, the U.S. Food and Drug Administration (FDA) is responsible for the regulation of clinical trial research using investigational products, including gene therapies for cancer indications. The FDA also regulates devices and combinations of therapy tools, drugs and/or devices. The regulatory rules may change over product categories as we learn more of the science risks and costs. While the invention disclosure is valid for its teachings everywhere, in the absence of regulatory approval in the US or other relevant jurisdiction, the skilled artisan is advised to confirm approvals including waste disposals and the like in practicing this invention.

### Example 1

A vesicle is created that distributes across a pH gradient. The excess time the vesicle resides at the lower pH increases its probability to decompose or release carried molecules. The rate of decomposition is sensitive to temperature resulting in a highly synergistic effect for delivering the effectors when both temperature and H⁺ are increased.

### Example 2

Probe T distributes according to temperature tending to bind lipid membranes as a function of a factor including, but not limited to: to fluidity, temperature dependent membrane protein access, intercellular access, etc. Probe H binds probe T only when probe T is protonated. Accordingly, at lower pH H-T binding is greatly increased. Stoichiometry may be 1:1 H:T or other relationship, e.g., 2:1,3:1,4:1, 3:2, 1:2, 2:3, 1:4, etc. Probe H or probe T or an activated chimer of the two may be activated to deliver metabolic modulation or other instruction or may serve as a binding agent for another effector agent.

What is claimed is:

1. A method of intercourse with a living cell, said intercourse comprising at least one activity selected from the group consisting of: identifying said cell, targeting said cell, segregating said cell, isolating said cell and triggering a natural death in said cell, said method comprising chemically or physically monitoring a population comprising one or more living cells, transmitting a signal reporting at least one characteristic selected from the group consisting of: a physical property and a chemical property, receiving said signal and correlating said signal with an indication of location of said cell.

2. The method of claim 1 wherein said intercourse comprises identifying, said identifying comprising monitoring at least two parameters.

3. The method of claim 2 wherein said at least two parameters comprise at least i) hydrogen ion concentration and ii) a temperature differential.

4. The method of claim 2 wherein said monitoring is carried out by a single component with sensitivity to at least two parameters.

5. The method of claim 4 wherein said single component monitors at least one physical property and at least one chemical property.

6. The method of claim 5 wherein said at least one physical property comprises temperature.

7. The method of claim 5 wherein said at least one chemical property comprises a concentration of at least one ion.

8. The method of claim 7 wherein said at least one ion comprises a hydrogen ion.

9. The method of claim 2 wherein said monitoring is carried out by a plurality of substances.

10. The method of claim 9 wherein at least one of said plurality of substances is delivered by a nanosensor.

11. The method of claim 9 wherein said at least two parameters comprise at least i) hydrogen ion concentration and ii) a temperature differential.

12. The method of claim 5 wherein said single component comprises a modified viral particle.

13. The method of claim 12 wherein said viral particle has been modified to increase cell interaction at increased hydrogen ion concentration.

14. The method of claim 12 wherein said viral particle has been modified to increase cell interaction at increased temperature.

15. The method of claim 14 wherein said viral particle comprises an engineered viral coat.

16. The method of claim 12 wherein said viral particle is selected from the group consisting of: vaccinia, lentiparticle, herpes, adenoparticle and reovirus.

17. The method of claim 16 wherein said viral particle comprises a herpes particle, said particle comprising a modified HSV-1.

18. The method of claim 9 wherein said plurality of substances comprises at least one first substance capable of binding at least one second substance, said at least one first substance monitoring at least one of said characteristic, said binding facilitating monitoring of at least a second characteristic.

19. The method of claim 9 wherein said plurality comprises a first and a second substance, said first and second substances cooperating to facilitate activity of at least one third substance.

20. The method of claim 19 wherein said at least one third substance is a natural substance endogenous to an organism hosting said living cell.

21. The method of claim 19 wherein said at least one third substance is delivered to an organism hosting said living cell.

22. The method of claim 19 wherein said activity comprises activating a composition comprising a component selected from the group consisting of: said at least one first substance, said at least one second substance, a reaction product of said at least one first substance, a reaction product of said at least one second substance, a membrane receptor exposed by said at least one first substance and a membrane receptor exposed by said at least one second substance.

23. A composition comprising at least one first substance capable of binding at least one second substance, said at least one first substance monitoring at least one first characteristic selected from the group consisting of: a physical property and a chemical property and at least one second substance monitoring at least one characteristic selected from the group consisting of: a physical property and a chemical property that differs from at least one first characteristic.

24. The composition of claim 23 wherein said binding is facilitated by a parameter selected from the group consisting of: H⁺ concentration or pH, O₂ concentration, aldehyde concentration, glucose concentration, lactate concentration, glutamine concentration, ketone concentration, CO₂ concentration, predetermined receptor presence and temperature.

25. The composition of claim 23 wherein said binding is sensitive to a parameter selected from the group consisting of: O₂ concentration and pH.

26. The composition of claim 23 further comprising at least one third substance capable of interaction with said at least one first substance, said at least one second substance, a reaction product of said at least one first substance, a reaction product of said at least one second substance, a membrane receptor exposed by said at least one first substance and a membrane receptor exposed by said at least one second substance.

27. A composition comprising at least two components, wherein at least a first component partitions in accordance with a H⁺ concentration in an organism and at least a second component partitions in accordance with temperature in said organism.

28. A composition comprising a lipid enclosed or enveloped particle, said particle designed to interact with a targeted cell at a temperature greater than the temperature normally ambient to said target cell.

29. The composition of claim 28 comprising a lipid enclosure wherein assimilation into a cell plasma membrane bears a positive correlation with increased temperature.

30. The composition of claim 27 wherein said lipid enclosed particle comprises a pH sensitive moiety selected to bind a plasma membrane target component on a cell whose local environment comprises a pH less than that of ambient cells.

31. The composition of claim 28 comprising an enveloped particle wherein assimilation into a cell plasma membrane bears a positive correlation with increased temperature.

32. The composition of claim 27 wherein said enveloped particle comprises a pH sensitive moiety selected to bind a plasma membrane target component on a cell whose local environment comprises a pH less than that of ambient cells.

33. The composition of claim 31 wherein said enveloped particle comprises an enveloped virus.

34. The composition of claim 33 wherein said enveloped particle is selected from the group consisting of: adenovirus, adeno-associated virus, lentivirus, vaccinia virus, herpes virus and reovirus.

35. The composition of claim 27, said composition comprising an inhibitor of protein expression.

36. The composition of claim 35 wherein said inhibitor of protein expression comprises an RNA.

37. The composition of claim 35 wherein said inhibitor of protein expression comprises a protein selected from the group consisting of: a polypeptide, a glycoprotein and a lipoprotein.

38. The composition of claim 35 wherein said inhibitor of protein expression comprises a transcription factor.

39. The composition of claim 38 wherein said transcription factor inhibits expression of at least one protein.

40. The composition of claim 39 wherein said at least one protein comprises a member selected from the group consisting of: an MCT, Drp1, Fis1, Mff, MiD49, MiD51, PGAM1, humanin, Bcl2, BcIXI, BcIxES, Nip3, P13K, Akt, mTOR, ACS, ACL, STAT1, Jak, Tyk2, CUG2, HGPRT, SETDB1, LDH1, GAC, GLS, GLS2, PAT1, GSG and fumarate dehydrogenase.
